(19) United States
(12) Patent Application Publication
(10) Pub. No.: US 2007/0287883 A1
(43) Pub. Date: Dec. 13, 2007

(54) Apparatus and method for pulling a cardiac harness onto a heart

(76) Inventors: Lilip Lau, Los Altos, CA (US); Joshua Wallin, San Jose, CA (US); Craig Mar, Fremont, CA (US)

Correspondence Address: FULWIDER PATTON LLP, HOWARD HUGHES CENTER, 6060 CENTER DRIVE, TENTH FLOOR, LOS ANGELES, CA, 90045, US

(21) Appl. No.: 11/448,593

(22) Filed: Jun. 7, 2006

(51) Int. Cl. A61F 2/00; A61F 13/00

(52) U.S. Cl. 600/37

(57) ABSTRACT

A delivery device configured for pulling a cardiac harness onto the heart is disclosed. The device includes a plurality of pull rods, a housing, and a control mechanism. The invention also includes a method of pulling a cardiac harness onto a heart. The method includes inserting the delivery device through a minimally invasive incision below the heart. Lines are connected with the cardiac harness and disposed over a distal end of a plurality of pull rods. The lines are pulled by the surgeon to mount the harness on the heart.

### FIELD OF THE INVENTION

The present invention relates generally to a device and method for delivering a cardiac harness onto the heart of a patient.

### BACKGROUND OF THE INVENTION

Congestive heart failure (“CHF”) is characterized by the failure of the heart to pump blood at sufficient flow rates to meet the metabolic demand of tissues, especially the demand for oxygen. It has been determined that a passive wrap, or cardiac harness may increase the efficiency of a heart affected by congestive heart disease. While advances have been made in cardiac harness technology, a satisfactory device and method for delivering and positioning the cardiac harness onto a patient's heart has yet to be provided.

In one method, access to a patient's heart is achieved through an open chest procedure, wherein the sternum is split and separated to allow access to the heart. The cardiac harness is then positioned over the heart by manual manipulation. Such an open chest procedure is highly traumatic to the patient and, thus, remains a relatively undesirable option for cardiac harness delivery.

Some present cardiac harness delivery devices do not both adequately retain the cardiac harness onto the delivery device and permit the harness to be easily released from the delivery device. For example, one delivery device utilizes sutures positioned around a circumference of the cardiac harness to secure it to the delivery device. Such arrangements render the cardiac harness difficult to release from the delivery device, especially on the rearward side of the heart. This is because the sutures have to be severed in order to release the cardiac harness from the delivery device. Such an arrangement would not be well suited for a minimally invasive procedure because an additional instrument would have to be introduced to sever the sutures.

At least one present delivery device uses push arms configured to connect with a cardiac harness. The harness is attached to the push arms, and the push arms are then pushed or advanced up around the heart. When the cardiac harness is mounted on the heart, the delivery device is then detached, leaving the harness implanted on the heart. One problem with the existing delivery systems is that the push arms must meet several critical mechanical engineering criteria. The push arms must be flexible and atraumatic in order to advance into the pericardial space and not damage the heart. At the same time, the push arms must be sufficiently stiff to transmit enough force to advance the push arms and the cardiac harness into the proper position.

Furthermore, a delivery system utilizing a plurality of push arms for the delivery of the cardiac harness does not work well in all patients. For example, it has been observed that in some patients the shape of the cardiac anatomy may be more globular or spherical. Also, in some patients the pericardium may be very tightly held to the epicardium, or there may be little space between the heart and the chest wall. In these circumstances, the push arms are forced to negotiate a very tight bend (small radius) in order to get into the pericardial space. In bending sharply, the ability of the push arm to transfer the pushing forces required to advance the cardiac harness onto the heart is significantly reduced. This is because the force is not efficiently transmitted through the tight bend.

Push arms work by transmitting force from the proximal end to the distal end of the push arm. The push arm must be flexible enough to negotiate the anatomy. The push arm must be capable of advancement over the heart atraumatically. Therefore, push arms may not be rigid enough to efficiently transmit the forces needed to push a cardiac harness onto the heart in all patients.

Pulling a cardiac jacket into place through a multitude of separate incisions in the chest has been considered in the art. One method describes making several incisions in the chest wall superior to the heart, making an additional incision in the chest wall for an endoscope, delivering the cardiac device through yet another subcostal incision, and pulling the cardiac device into place. The cardiac harness or jacket has multiple strands attached to its base. The cardiac jacket is pulled from above the heart by forceps, graspers, or pickups which must first find and grasp each of the strands attached to the base of the harness. This method has several disadvantages. Placing multiple incisions in the chest wall superior to the heart is traumatic to the patient, and runs the risk of producing a pneumothorax or injuring any number of vascular and nervous structures in the chest wall. Furthermore, passing grasping instruments from superior to the heart to the sutures located inferior to the heart runs a risk of injury to the great vessels, the lungs, or the heart itself. Finally, individually grasping each strand is tedious, technically challenging, dangerous, and time consuming.

It has therefore been recognized by those skilled in the art that in certain patients it is advantageous to pull a cardiac harness, cardiac jacket, or other pericardial implantable device into place rather than pushing the cardiac harness into place. It has also been recognized by those skilled in the art that a minimally invasive subcostal or subxiphoid incision, without the need for supracardiac chest wall incisions, results in less risk to the patient, is less traumatic, and is conducive to a faster recovery and a shorter hospitalization at less expense. Furthermore, it has been recognized by those skilled in the art that there is a need for a device that conveniently and expeditiously pulls the cardiac harness onto the heart, for example, through one or more subcostal incisions. It has also been recognized by those skilled in the art that there is need for a delivery device may be used to mount a cardiac harness on the heart, using a pulling action on the harness, with a subcostal or subxiphoid incision, and without the need for multiple chest wall incisions superior to the heart. The present invention addresses these needs and others.

### SUMMARY OF THE INVENTION

The present invention addresses the need to deliver a cardiac harness by pulling the cardiac harness onto the heart, for example, from a subcostal incision. The present invention may be advantageous where the heart is somewhat spherical and the initial bend to be navigated in placing the harness on the heart is quite severe. Briefly and in general terms, the invention is a cardiac harness delivery device that is configured to support the cardiac harness in a compacted configuration to permit minimally invasive delivery of the cardiac harness onto the heart with a pulling action. The cardiac delivery device is configured to deliver the harness onto a more spherically shaped heart through a minimally invasive procedure. The delivery device may be used to mount the cardiac harness on the heart, using a pulling action on the harness, with a subcostal or subxiphoid incision, and without the need for multiple chest wall incisions superior to the heart.

In one aspect of the invention, the delivery device includes pull rods that are configured to be deployed into the pericardial space. In yet another aspect of the invention, the distal ends of the pull rods are configured to connect with lines. In still another aspect of the invention, the lines may be detachably connected with the pull rods. The lines may be connected with an implantable cardiac harness. The lines may be detachably connected with an implantable cardiac harness. In a further aspect of the invention, the distal ends of the pull rods may have friction reducing members disposed on them. The friction reducing members may assist the lines in sliding upon the ends of the pull rods. Pulling on the free ends of the lines will pull the harness into position on the heart. After positioning the harness upon the heart, the lines may be detached from the harness and/or the pull rods. The pull rods may then be retracted off of the heart, leaving the harness mounted in place on the heart.

Another aspect of the present invention includes a delivery device for use with pulling a cardiac harness onto a heart including a handle slidingly connected with a proximal end of an elongate shaft. A housing may be affixed to a distal end of the elongate shaft. The housing has a housing cavity within which a cardiac harness may be held in a compacted configuration. A plurality of pull rods capable of sliding axial movement with the housing may be connected proximally with the handle. The pull rods may be capable of a sliding connection with lines that may be disposed over the distal ends of the pull rods. In yet another aspect, the invention further includes at least one friction reducing member connected with the distal portion of the pull rod. The friction reducing members may be included in rod heads disposed on the distal ends of the pull rods. In yet one more aspect, the invention further includes lines capable of a releasable connection with the cardiac harness. In still another aspect, the invention further includes line guides in the wall of the housing. In yet another aspect, the invention further includes a cardiac harness releasably connected with lines, the lines being disposed on the friction reducing members that are connected with the distal portion of the pull rods. In yet another aspect of the invention, the device further includes shaped channels in the housing for slidingly connecting with and retaining the pull rods. For example, the channels in the housing may be dove tail shaped cut outs or recesses that articulate with pull rods that are at least partially dove tail shaped in cross section. In still a further aspect of the invention, the delivery device may include a pull rod slider for retaining the pull rods. In yet one more aspect of the invention, the lines or cables may be connected with a cable slider configured for simultaneously pulling on the plurality of lines or cables in unison.

In one aspect of the invention, the device includes pull rods that are contoured to fit into the pericardial space. The pull rods may include a hollow inner channel for passage of at least one line therethrough. In yet one other aspect of the invention, pull rods include a side opening extending the length of the hollow inner channels for insertion of the lines through the inner channel. The lines may be detachably connected with a harness. In still one further aspect of the invention, one or more of the pull rods may be retained in a pull rod carrier.

Yet another aspect of the invention includes a method of disposing a cardiac harness onto the heart including making an incision in a patient adapted to access the heart. An incision is also made in the pericardium. The distal end of the delivery device is introduced into the patient through the incision. A plurality of pull rods may be advanced over the surface of the heart between the epicardium and the pericardium, until the pull rods are disposed around the heart. Pulling on a plurality of lines connected to a cardiac harness advances the harness towards the distal ends of the pull rods. The lines are pulled until the harness is in a desired location surrounding the heart. In another aspect of the invention, the lines are configured to be releasably attached with the harness. The lines may be double lines that are formed by passing a line through a part of the harness, for example, an undulation in the harness, and doubling the line back on itself. The doubled line is configured to form a loop connected with the harness distally, and two free ends of line proximally. Simultaneously pulling on both of the free ends of a doubled line advances the harness toward the distal end of the pull rod. Pulling on one free end of the doubled line detaches the line from the harness. After the harness is detached from the line, the pull rods may be removed from around the heart, thereby leaving the harness in place on the heart. The delivery device may then be removed from the patient and the incision closed by the surgeon.

In still another aspect of the invention, the method of disposing a cardiac harness onto the heart of may further include releasably connecting a plurality of lines around the periphery of the harness and loading the cardiac harness into a housing cavity of the cardiac harness delivery device. The lines may be doubled lines. The lines disposed on the harness are then slidingly disposing onto the distal portions of the pull rods. The free ends of the lines may then be brought back to the proximal end of the cardiac harness delivery device.

In a further aspect of the invention, the method of disposing a cardiac harness onto the heart may further include slidingly connecting the lines with line guides in the housing. In yet another aspect of the invention, the method includes disposing the line onto the distal portion of at least one pull rod by slidingly disposing the line on a friction reducing member. In still another aspect of the invention, the method includes closing the pericardium after disposing the harness onto the heart, thereby reducing epicardial scarring. In yet another aspect of the invention, the method includes preloading of the cardiac harness and the lines onto the delivery device, for example by the manufacturer, prior to delivery to the clinician.

In another aspect of the invention, the method of implanting the cardiac harness onto the heart includes pulling a cardiac harness towards the distal portion of at least one pull rod. The cardiac harness may be pulled towards the distal portion of the pull rod using a line or a doubled line. For example, a loop in the doubled line may be slidingly connected with the harness. The doubled line may then be slidingly disposed on the distal end of the pull rod, for example by passing the doubled line over or through a friction reducing member on the distal end of the pull rod. The friction reducing member may be included on a rod head disposed on the distal end of the pull rod. The pull rod is inserted into the pericardial space. The cardiac harness is implanted onto the heart by pulling on the doubled line. The cardiac harness may be detached from the pull rod, for example, by pulling on only one of the free ends of the doubled line, thereby permitting the line to slide through the harness. The pull rod may be withdrawn from the pericardium, leaving the harness implanted on the heart. In one aspect of the invention, the method may further include disposing the pull rods onto a pull rod carrier for holding and organizing the proximal ends of the pull rods.

In still another aspect of the invention, the method further includes disposing the lines over the distal end of a pull rod, connecting at least one end of the line with the harness and connecting the other end of the line to the cable slider. In yet another aspect, the invention includes slidingly connecting at least one line with a friction reducing member that is disposed on the distal portion of the pull rod, for example in a rod head. In yet one further aspect of the invention, the method may also include disposing at least one line through an inner channel in the pull rod.

### BRIEF DESCRIPTION OF THE DRAWINGS

These and other features, aspects and advantages of the present invention are described with reference to drawings of a preferred embodiment, which are intended to illustrate, but not to limit, the present invention.

FIG. 1 is a perspective view of a delivery device of the invention.

FIG. 2 is an enlarged, partial cutaway view of a distal portion of the delivery device of FIG. 1.

FIG. 3 is a perspective view of the delivery device of FIG. 1.

FIG. 4 is a cross sectional view through a housing.

FIG. 5A is an enlarged perspective view of the delivery device of FIG. 1 showing distal ends of a plurality of pull rods.

FIG. 5B is an enlarged plan view of a distal portion of a pull rod showing an embodiment wherein a friction reducing member includes a bushing.

FIG. 5C is an enlarged perspective view of a distal portion of a pull rod showing another embodiment of a friction reducing member.

FIG. 5D is an enlarged side view of a distal portion of a pull rod showing another embodiment of a friction reducing member.

FIG. 6A shows a line threaded through one loop of an undulating row of a harness.

FIG. 6B shows a line threaded through two loops of an undulating row of a harness.

FIG. 6C shows a line threaded through three loops of an undulating row of a harness.

FIG. 6D shows lines threaded through intermediary connecting members.

FIG. 7 illustrates a cardiac harness that is reinforced by struts having reinforced eyelets.

FIG. 8 illustrates a cardiac harness disposed in a housing cavity with lines disposed in friction reducing members.

FIG. 9 schematically shows insertion of the delivery device of FIG. 1 into a patient in a minimally invasive surgery.

FIG. 10 schematically advancement of a cardiac harness over a heart of a patient using the delivery device of FIG. 1 in a minimally invasive surgery.

FIG. 11A-C schematically illustrates how a cardiac harness may be pulled onto a heart and detached from lines.

FIG. 12 shows removal of the delivery device of FIG. 1 in a minimally invasive surgery.

FIG. 13 is an enlarged side view of a distal end of a pull rod.

FIG. 14 is an enlarged side view of a control assembly of the delivery device of FIG. 1.

FIG. 15 is a cross sectional view of a body portion of the control assembly of FIG. 14.

FIG. 15A is a cross sectional view of an embodiment of a pull rod.

FIG. 16 is a longitudinal cross-sectional view of the control assembly of FIG. 14.

FIG. 17 is a longitudinal cross-sectional view of the control assembly of FIG. 14.

FIG. 18 is a perspective view of an embodiment of a contoured pull.

FIG. 19 is a longitudinal sectional view of the pull rod of FIG. 18.

FIG. 19A is a cross sectional view of the pull rod of FIG. 18.

FIG. 20 is a perspective view of a carrier for pull rods.

FIG. 21 is an illustration of an embodiment of a cardiac harness including electrodes and reinforced eyelets connected with an ICD.

FIG. 22 is a perspective view of an embodiment of a cardiac harness delivery device in an undeployed first configuration.

FIG. 23 is a perspective of the cardiac harness delivery device of FIG. 22 in a fully deployed third configuration.

FIG. 24 is an enlarged view of the cardiac harness delivery device of FIG. 22 showing a section through a rod head.

FIG. 25 is a perspective of the cardiac harness delivery device of FIG. 22 in an intermedate second deployed configuration.

FIG. 26 is a perspective view through a cable slider of the cardiac harness delivery device of FIG. 22.

### DETAILED DESCRIPTION OF THE PREFERRED EMBODIMENTS

The present invention includes a cardiac harness delivery device useful for pulling a cardiac harness onto a heart. The present invention is a new and improved device and method of delivering the harness onto the heart, related to U.S. Ser. No. 10/715,150 entitled “Cardiac harness delivery device and method” filed on Nov. 17, 2003, the entirety of which is incorporated herein by reference.

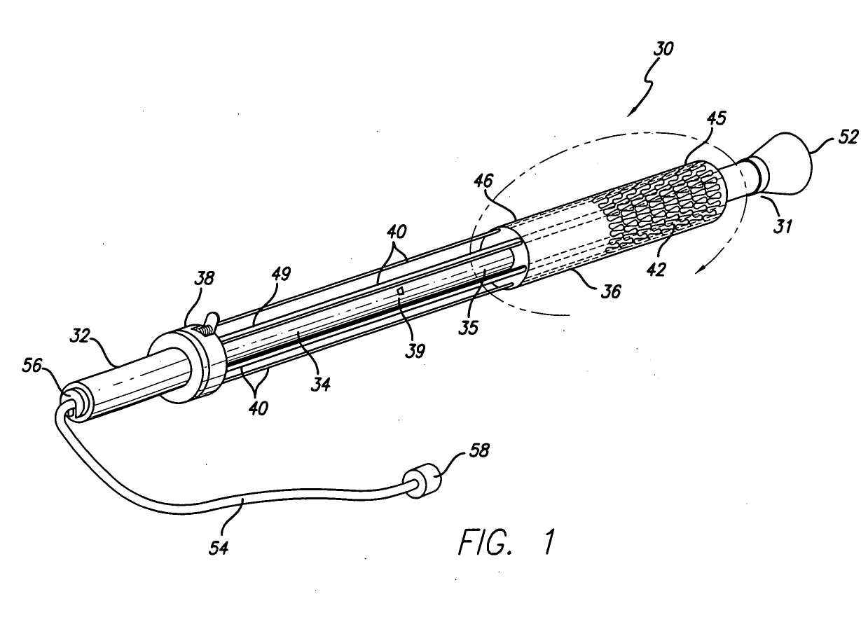

Referring to the drawings, which are provided for purposes of illustration and by way of example, FIG. 1 illustrates an embodiment of a cardiac harness delivery device, which is generally referred to by the reference numeral 30. In a preferred embodiment, the delivery device 30 is configured to releasably deliver and advance a cardiac reinforcement device (CRD), such as a cardiac harness 42, over the heart of a patient and mount the cardiac harness on the heart. The cardiac harness 42 is advanced over the heart by a pulling action on the cardiac harness towards the heart. Once the cardiac harness is mounted on the heart, the delivery device 30 is configured to release the cardiac harness on the heart. The delivery device may then be retracted from the patient without causing undesired shifting of the cardiac harness relative to the heart.

In the illustrated arrangement, the delivery device 30 permits delivery of a cardiac harness in a minimally invasive manner. That is, preferably the device 30 permits accurate delivery, positioning, and release of the cardiac harness through a minimally invasive procedure in a patient. However, embodiments of the delivery device 30 may also be used to deliver a cardiac harness in an open chest procedure. Furthermore, at least one embodiment is configured to enable direct or indirect visualization of at least portions of the device 30 during surgery. For example, portions of the device may be radiopaque or partially radiopaque so as to be visualized and guided by fluoroscopy or other methods. In yet another embodiment, the harness may be viewed endoscopically during delivery and mounting on the heart.

The illustrated delivery device 30 generally includes a body portion comprised of a handle 32 slidingly connected with the proximal end of a hollow, elongate shaft 34. A proximal end or base portion 46 of a housing 36 is affixed to a distal end 35 of the elongate shaft 34. The illustrated delivery device 30 also includes a movable portion comprised of a control assembly 38 and a plurality of elongate pull rods 40. The control assembly 38 is longitudinally slidable along the shaft 34, and, thus, the pull rods 40 are longitudinally slidable in relation to the shaft 34 and housing 36.

The handle 32 is fixed to the shaft 34 in the illustrated embodiment. However, it is to be understood that in other arrangements the handle 32 may be movable relative to the shaft 34 along with the control assembly 38. Additionally, another embodiment may not employ a handle 32. A stop 39 may be provided on the shaft 34 in some embodiments. The stop 39 comprises a raised portion that engages the control assembly 38 so that the assembly 38 cannot move distally over the shaft 34 beyond the stop 39. However, the stop 39 is not necessary in all embodiments.

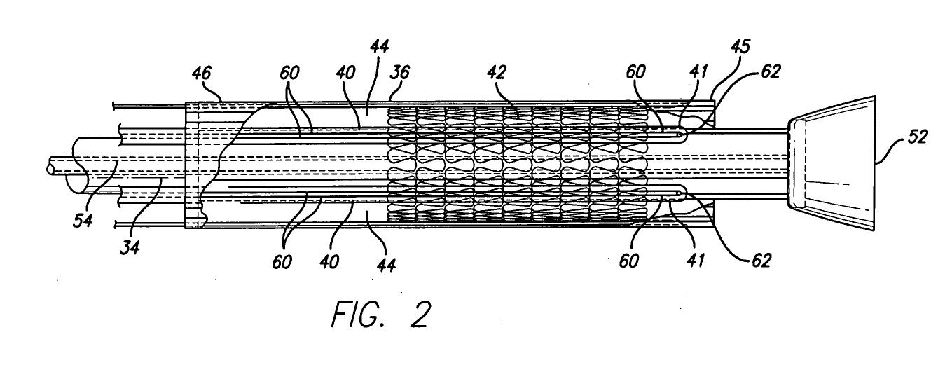

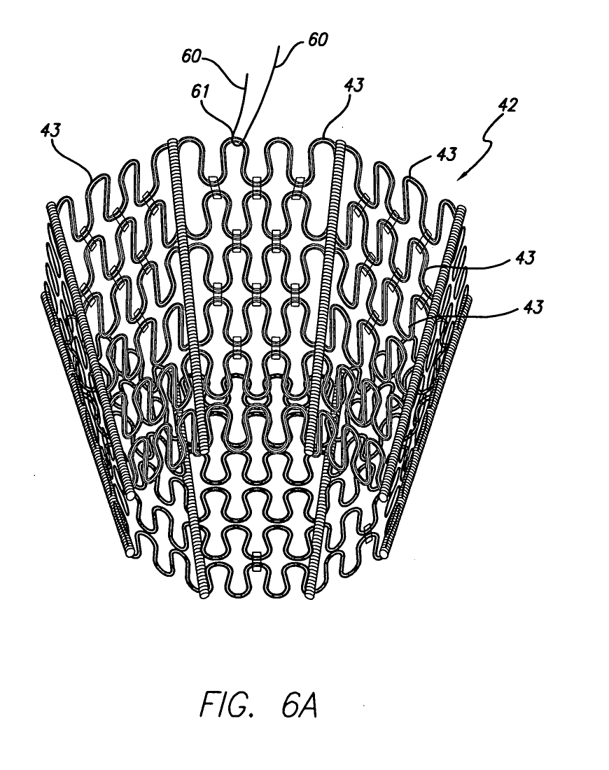

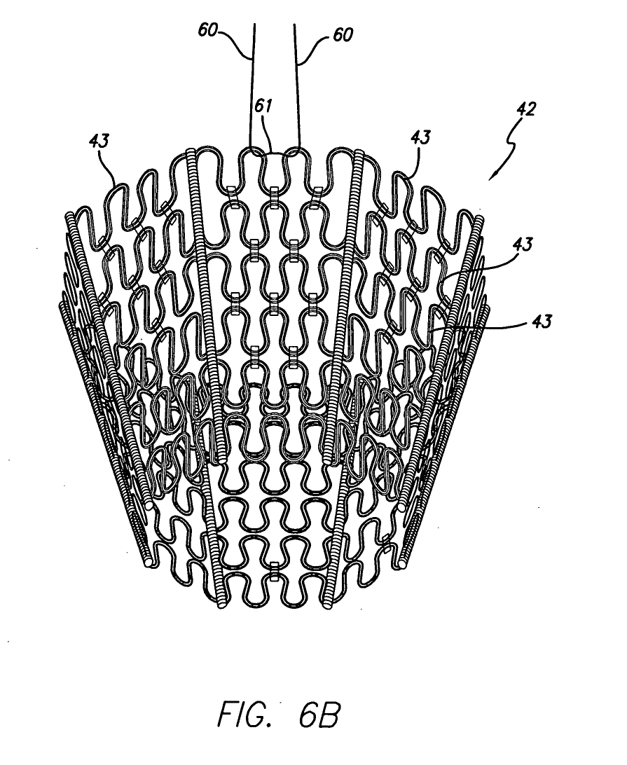

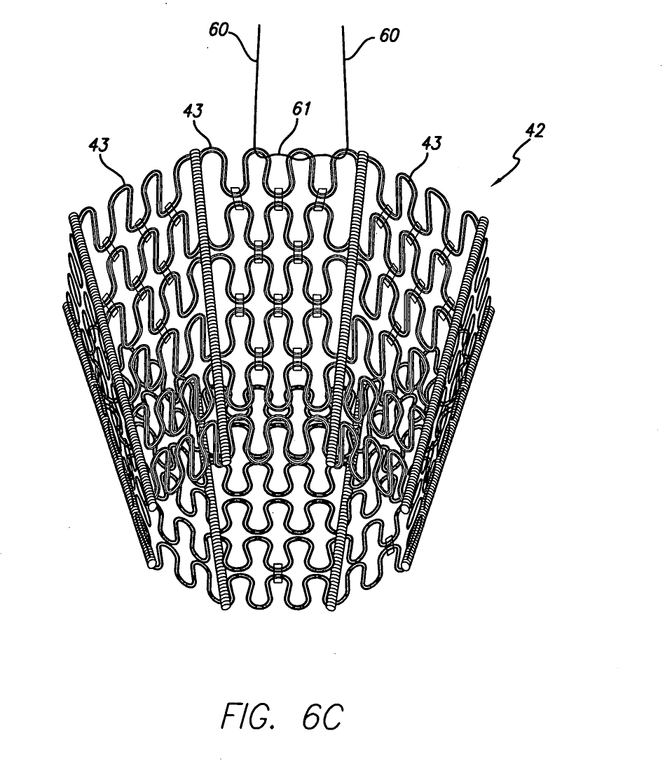

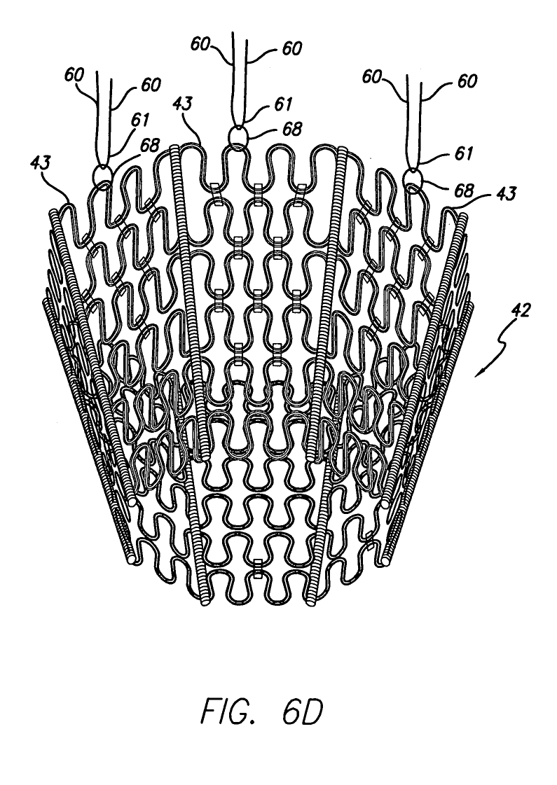

With reference also to FIG. 2, a cardiac harness 42 is releasably held in a compacted configuration within a housing cavity 44 in the housing 36. Preferably, the cardiac harness 42 is configured to fit around the heart and to exert a compressive force on the heart. In the illustrated embodiment, the harness 42 comprises several interconnected rows of undulating elastic members 43 (FIG. 6A). Preferred cardiac harnesses are described in greater detail in U.S. Ser. No. 09/634,043, filed Aug. 8, 2000 now U.S. Pat. No. 6,702,732; U.S. Ser. No. 10/242,016, filed Sep. 10, 2002 now U.S. Pat. No. 6,723,041; U.S. Ser. No. 10/287,723, filed Oct. 31, 2002; and U.S. Ser. No. 10/656,722, filed Sep. 5, 2003, the entirety of each of which are incorporated by reference herein. It is to be understood that aspects of the delivery device 30 discussed herein can be used in connection with several other types of cardiac harnesses or jackets.

The term “cardiac harness” as used herein is a broad term that refers to a device adapted to fit onto a patient's heart to apply a compressive force on the heart during at least a portion of the cardiac cycle. The meaning of “cardiac harness” includes a device that is intended to be disposed onto and reinforce a heart, and which may be referred to in the art as a “girdle,” “sock,” “jacket,” “CRD,” or the like.

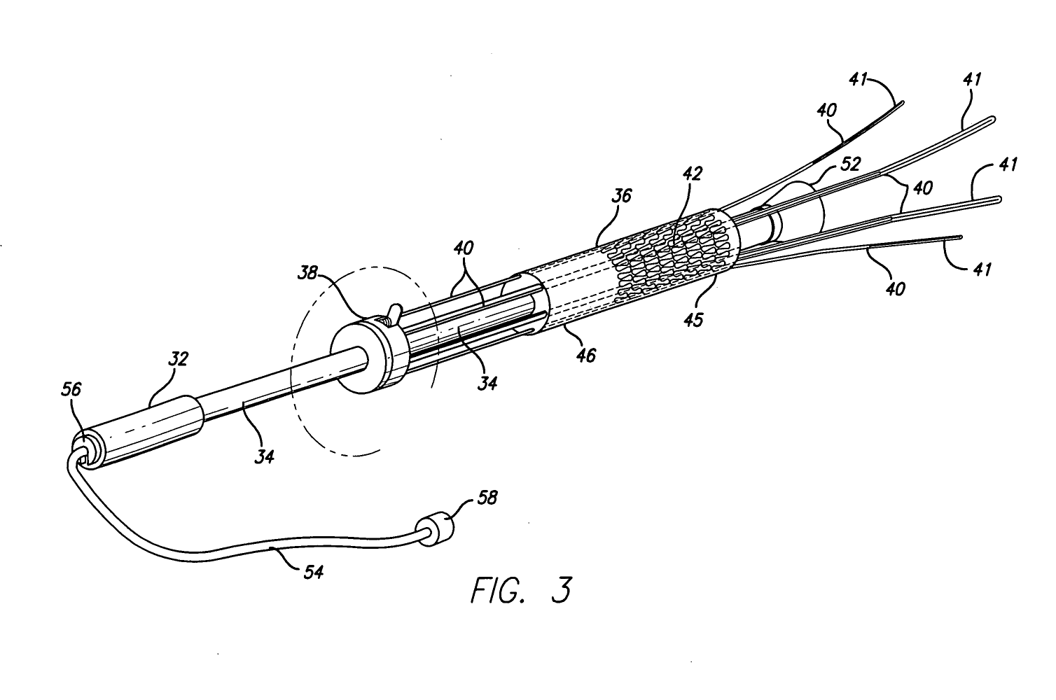

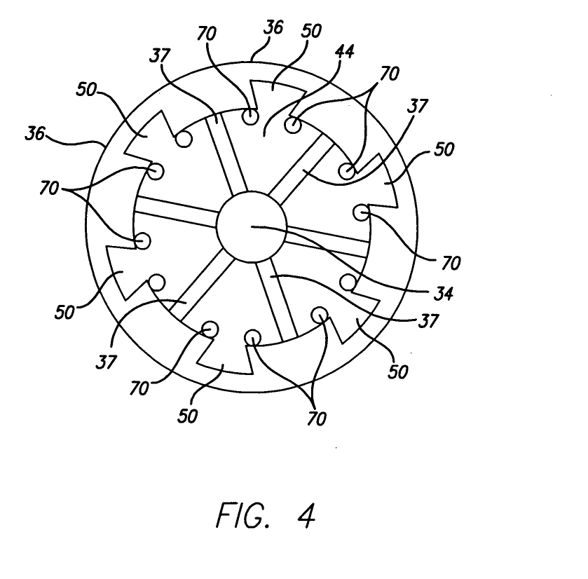

Referring also now to FIG. 3 and FIG. 4, the plurality of pull rods 40 extend in a distal direction from the control assembly 38 and pass through, or alongside, the housing 36. The pull rods 40 are elongated and substantially flat members. The pull rods 40 have distal ends 41 that may be blunted, substantially flexible, and/or divergent from the center axis of the delivery device so as to avoid trauma to the heart. In at least one embodiment, the pull rods 40 are dovetail shaped or substantially trapezoid shaped in cross section, and are capable of forming a sliding axial connection with corresponding dovetail shaped channels 50 in the housing 36. The housing 36 is axially slidable along the pull rods 40, and in at least one embodiment, transversely secured by the dovetail connection of each pull rod 40 with the housing 36. The dovetail connection of each pull rod 40 to the housing 36 permits each pull rod 40 to move axially and prevents the pull rods 40 from be dislocated radially inwardly or outwardly from the housing 36. Other shapes that slidingly retain the pull rods 40 in the channels 50 may also be used. In yet other embodiments, clips or rings may be used to retain the pull rods 40 in proximity to the wall of the housing 36.

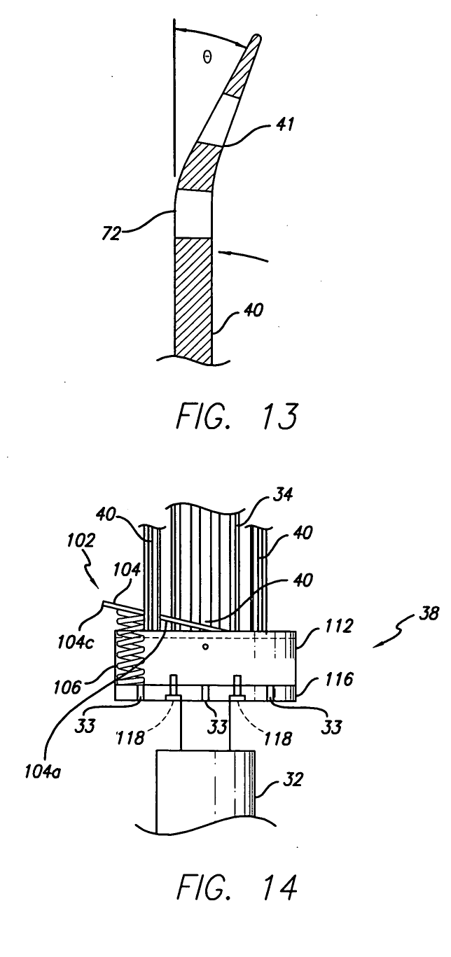

Referring briefly now also to FIG. 13, although preferably the inner surface 72 of each pull rod 40 is generally planar in a relaxed orientation, the pull rod 40 is configured to be deflectable so as to splay outwardly from a distal end 45 of the housing 36 so as to conform to an outer surface of a patient's heart while in use. Accordingly, the pull rod 40 is not always oriented such that the inner surface 72 is necessarily planar. However, when the pull rod 40 is in a splayed orientation, any given point on the surface preferably is either the same perpendicular distance from a center axis of the shaft 34, or a greater distance, than any point on the inner surface of the pull rod 40 proximal to the given point. That is, preferably, the inward facing surface 72 of the pull rod 40 does not have any inwardly extending portions when moving from a proximal end of the pull rod 40 toward a distal end 41 of the pull rod 40.

Referring again to FIG. 1 and FIG. 3, the control assembly 38 and plurality of pull rods 40 are movable axially with respect to the shaft 34 from the retracted position illustrated in FIG. 1 to an advanced, or deployed position, as illustrated in FIG. 3. Thus, the delivery device 30 is configured to deploy the pull rods 40 from a compacted substantially parallel configuration within the housing 36 to an expanded divergent configuration outside of the housing 36. As the pull rods 40 are advanced out of the housing 36, the rods assume the divergent configuration outside of the housing 36, and the pull rods 40 are capable of sliding upwardly over the heart, between the epicardium and the pericardium. The cardiac harness 42 may thereafter be pulled and guided onto a heart using the pull rods 40, as is described in greater detail below.

With reference again to FIGS. 2 and 4, the housing 36 preferably is a relatively thin-walled, tubular member. Desirably, the housing 36 is supported substantially concentric with the shaft 34. In at least one embodiment, the proximal end or base portion 46 of the housing 36 is affixed to the elongate shaft 34 by housing support members 37. The wall of the housing 36 defines an interior cavity 44 therein. In at least one embodiment, the cavity 44 is sized and shaped to contain the cardiac harness 42 in a compacted configuration therein. In at least one embodiment, the housing 36 is made of a substantially transparent material to aid in the visualization of the deployment of the harness during a surgical procedure.

In at least one embodiment, the delivery device 30 is configured to deliver the cardiac harness 42 in a minimally invasive procedure. Accordingly, a preferred housing 36 has a nominal outer diameter of less than about two inches and, more preferably, less than about one and one-half inches. However, in additional, non-minimally invasive embodiments, the housing 36, if provided, may be larger than the values given above. In some embodiments, the harness 42 may be supported by the device 30 in a configuration substantially similar to the configuration of the harness 42 when positioned on a heart. That is, the cardiac harness does not have to be supported in a “compacted” configuration by the device, but may be supported in a configuration closer to its relaxed size and shape.

In the embodiment shown in FIGS. 1-3, the housing 36 is generally cylindrical. It is to be understood that, in another preferred embodiment, the housing is elliptical. As such, the housing may have a major axis and minor axis. This configuration may be especially beneficial for advancing the housing through body passages having relatively narrow clearance, such as advancing the housing between the ribs.

With reference to FIG. 2 and FIG. 4, a proximal end or base portion 46 of the housing 36 preferably defines a closed end of the cavity 44 and supports the housing 36 relative to the shaft 34. The proximal end or base portion 46 of the housing 36 may be secured to the shaft 34 by housing support members 37 including mechanical fasteners, adhesives or other suitable methods known to one of skill in the art. In one embodiment, the proximal end or base portion 46 is rotatable relative to the shaft 34. The distal end 45 of the housing cavity 44 is open to permit the cardiac harness 42 to be advanced from the cavity 44.

In at least one embodiment, a plurality of shaped channels 50 are included in the housing 36, extending axially throughout the length of the housing 36. Each of the channels 50 preferably is sized and shaped to slidably receive one of the plurality of pull rods 40, each pull rod having a corresponding cross section. In one embodiment the channels 50 are dovetail shaped in cross section and the pull rods are dovetail shaped in cross section. Each pull rod 40 is thereby transversely secured within a channel 50 by the dovetail articulation, while remaining axially slidable within the channel 50. In at least one embodiment, the number of channels 50 is equal to at least the number of pull rods 40. Further, in at least one embodiment, each channel 50 opens into the housing cavity 44 along at least a portion of the length of the channel 50.

In the exemplary embodiment, six pull rods 40 and channels 50 are provided and are substantially equally spaced around the circumference of the housing 36. In other embodiments, however, there may be a greater or lesser number of pull rods 40 and channels 50. Furthermore, in some embodiments the channels 50 may be omitted and the pull rods 40 may simply be restrained from moving radially outwardly by the wall of the housing 36 or other retaining members, for example, clips or rings (not shown). Other suitable arrangements to guide the pull rods 40 and house the cardiac harness 42 may also be used.

With reference to FIGS. 1-3, in at least one embodiment, the delivery device 30 preferably includes a positioning member 52 configured to hold the delivery device 30 in a desired orientation relative to the heart. In at least one embodiment, the positioning member 52 helps to hold the apex of the heart while deploying the pull rods 40 around the heart. The positioning member creates tension on the heart and may create an inferiorly oriented caudal force that opposes the superiorly oriented cephelad force generated during the pulling of the harness onto the heart. Furthermore, the positioning member is advantageous in pulling on the apex of the heart, thereby modifying the shape of the heart from a spherical configuration to a more bullet shaped configuration. In the illustrated arrangement, the positioning member 52 comprises a suction cup supported on a distal end of the shaft 34. In still other embodiments, the positioning member 52 may be a releasable clip or grasping instrument known in the art. In at least one embodiment, a tube 54 extends through the shaft 34 and is connected to the suction cup positioning member 52. A distal end of the tube 54 opens into an interior space defined by the positioning member 52. The proximal end of the tube 54 includes a connector 58 that allows connection of the tube 54 to a pump member such as a syringe or other source of vacuum or negative atmospheric pressure. Accordingly, once the delivery device 30 is properly positioned, air may be withdrawn from within the tube 54 to create a vacuum condition within the interior space of the suction cup positioning member 52, thereby permitting the suction cup positioning member 52 to securely hold the apex of the heart of a patient.

Furthermore, in at least one embodiment, a clip 56 secures the tube 54 relative to the handle 32 to prevent the proximal end of the tube 54 from passing through the shaft 34. Thus, the clip 56 also operates in some embodiments to secure the suction cup positioning member 52 to the delivery device 30. In a preferred embodiment, the tube 54 and suction cup positioning member 52 are not rigidly affixed to the shaft 34 so that the shaft 34 may be moved relative to the tube 54 and suction cup positioning member 52. In another embodiment, the shaft 34 and a proximal end of the suction cup positioning member 52 are threaded so that the suction cup positioning member 52 may be threaded onto the shaft 34. In still other embodiments, other structure may be used to releasably connect the positioning member 52 to the shaft 34.

In at least one further embodiment, the shaft 34 is open on both of its ends. An endoscope or other fiber optic instrument may be inserted into or through the shaft 34 to aid in visualization of the heart and other internal anatomy. In addition, the shaft 34 can include a light source (not shown) to aid the surgeon in visualizing a patient's anatomy. Furthermore, a shaft that is at least partially open on both ends allows for irrigation fluid to be injected through the shaft 34, thereby reducing the friction between the delivery device 30 and the patient's anatomy, thereby aiding in the sliding of the harness 42 over the heart during delivery.

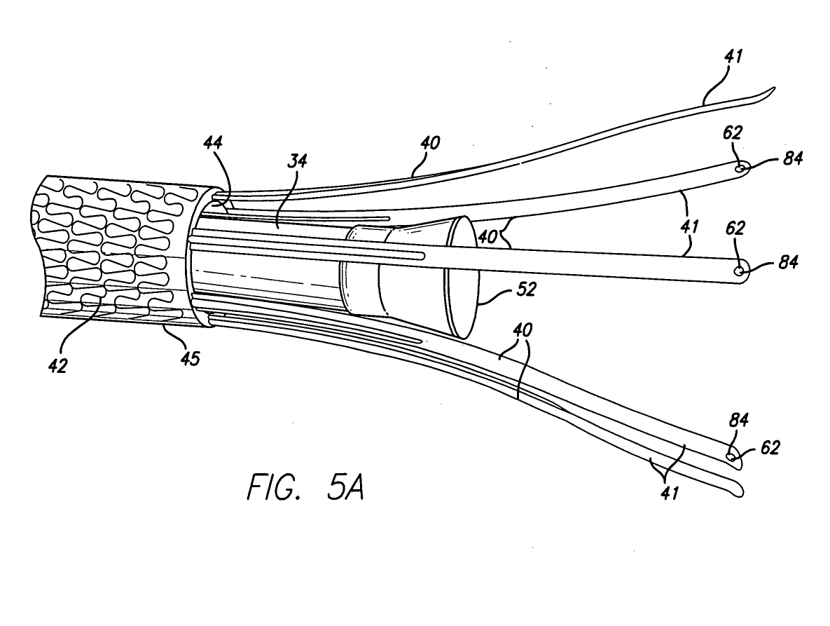

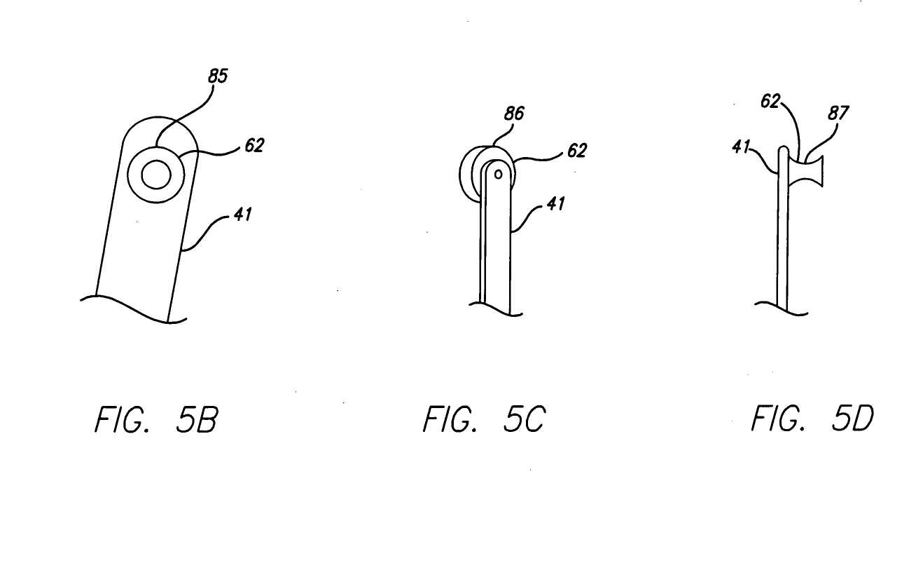

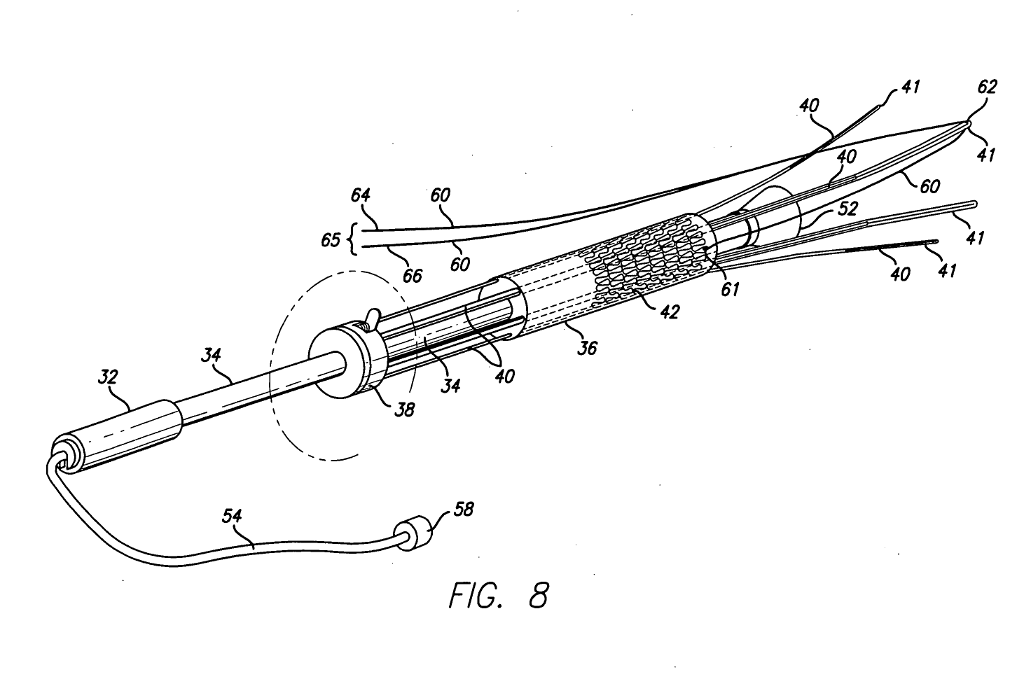

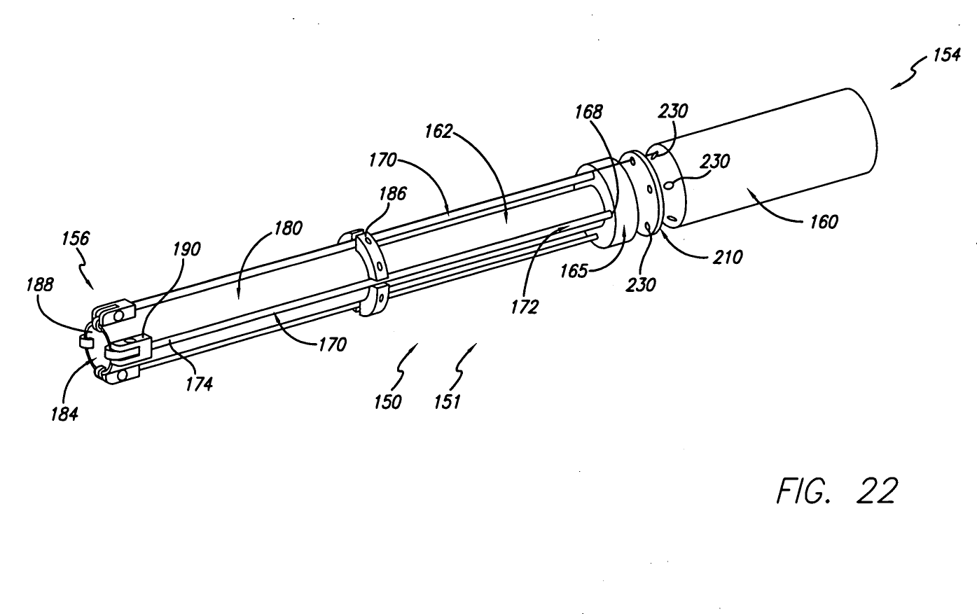

With reference next to FIG. 5A and FIG. 8, the distal end portion 41 of each of the plurality of pull rods 40 may be slidingly connected with a line 60. In some embodiments, the distal end portion 41 of each of the plurality of pull rods 40 includes a friction reducing member 62 capable of a sliding connection with the line 60. In some embodiments, the lines 60 are treated or coated with a friction reducing material, for example polytetrafluoroethylene (PTFE). As shown in FIG. 5A, the friction reducing member 62 may in one embodiment be a hole 84 in the distal end 41 of the pull rod 40. In one embodiment, the hole may be coated with a friction reducing material, for example, a biocompatible material having a low coefficient of friction. As shown in FIG. 5B, in at least one embodiment the friction reducing member 62 may be a bushing 85 having a low coefficient of friction. As shown in FIG. 5C, the friction reducing member 62 in yet another embodiment may be a pulley 86. As shown in FIG. 5D, in still a further embodiment, the friction reducing member 62 may be a substantially cylindrical drum 87 having a tapered middle section and/or a surface channel for a line 60 to slide thereon. The friction reducing member 62 may be coated with a biocompatible material having a low coefficient of friction, for example polytetrafluoroethylene (PTFE) (TEFLON) or polyoxymethylene (DELRIN). The friction reducing member 62 may also be included in a rod head 190 (FIG. 22-25) as further described below. The above examples are not meant to be limiting, and other configurations, shapes, and materials for reducing friction between a friction reducing member 62 and a line 60 will be known to those skilled in the art.

Referring again now to FIG. 8, the lines 60 may be one or more biocompatible strings, strands, filaments, cables, cords, or fibers, for example, sutures or flexible metal wires. The line 60 in at least one embodiment is a bioabsorbable suture with enough tensile strength to withstand the pulling forces applied to it when delivering the harness 42, without the line 60 breaking. Other suitable substantially thin and flexible materials suitable for the lines 60 are known in the art. In at least one embodiment, each line 60 is doubled over itself to form a loop 61 on the distal portion of the doubled line 60 and two free ends 64, 66 on the proximal portion of the doubled line.

Referring now also to FIGS. 6A-6D, the lines 60 may be connected with the cardiac harness 42 by threading one free end 64 or 66 of each of the lines 60 through one or more of the undulating elastic members 43 of the harness 42. The lines 60 are preferably placed around the periphery of the base of the harness 42. The base of the harness is that end of the harness that will be located at the superior aspect of the heart after implantation of the harness. After passing one free end 64 or 66 (FIG. 8) of the line 60 through the harness 42, the line 60 is then doubled back upon itself. Each line 60 when doubled back upon itself will thereby form a loop 61 at the distal portion of the doubled line, connected with the cardiac harness, and a pair of free ends 64 and 66 at the proximal portion of the doubled line. Furthermore, In some embodiments, the line 60 may be threaded through one or more of the undulations of the rows 43 in the harness 42. In other embodiments, the loops 61 in the lines 60 may be slidingly connected with intermediary connecting members 68, for example rings or clips, that are disposed around the periphery of the cardiac harness 42.

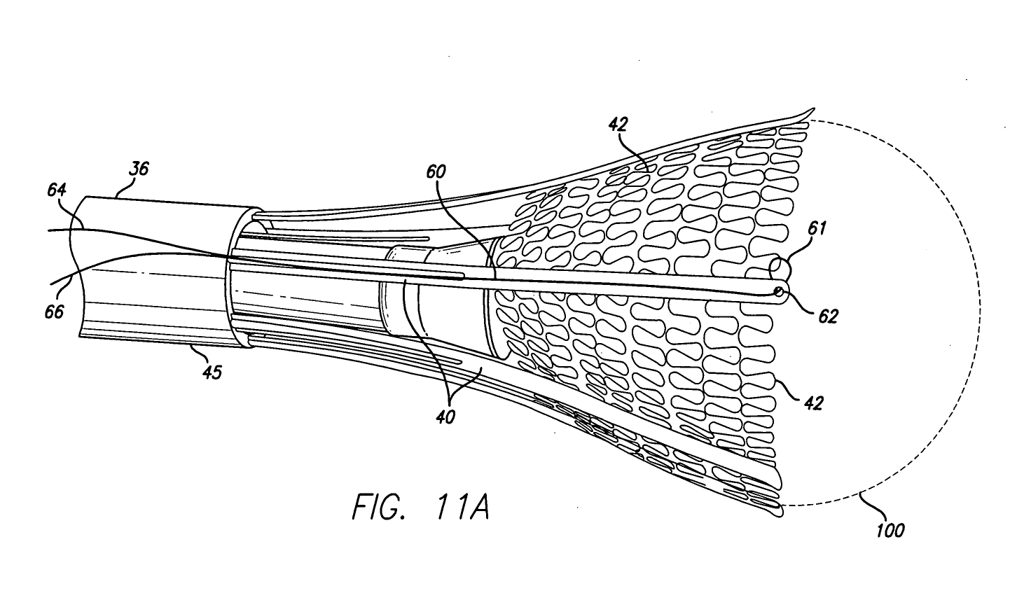

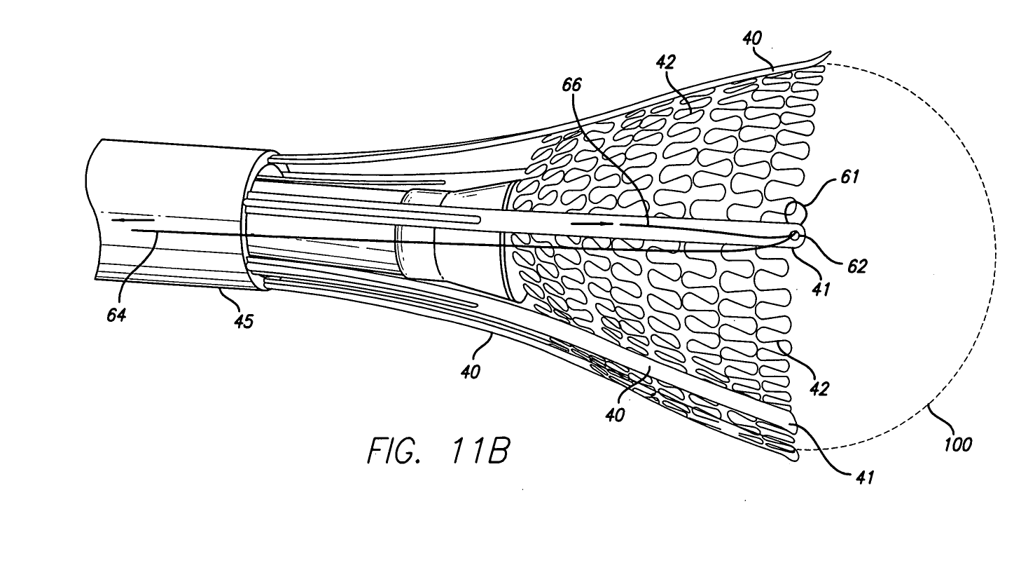

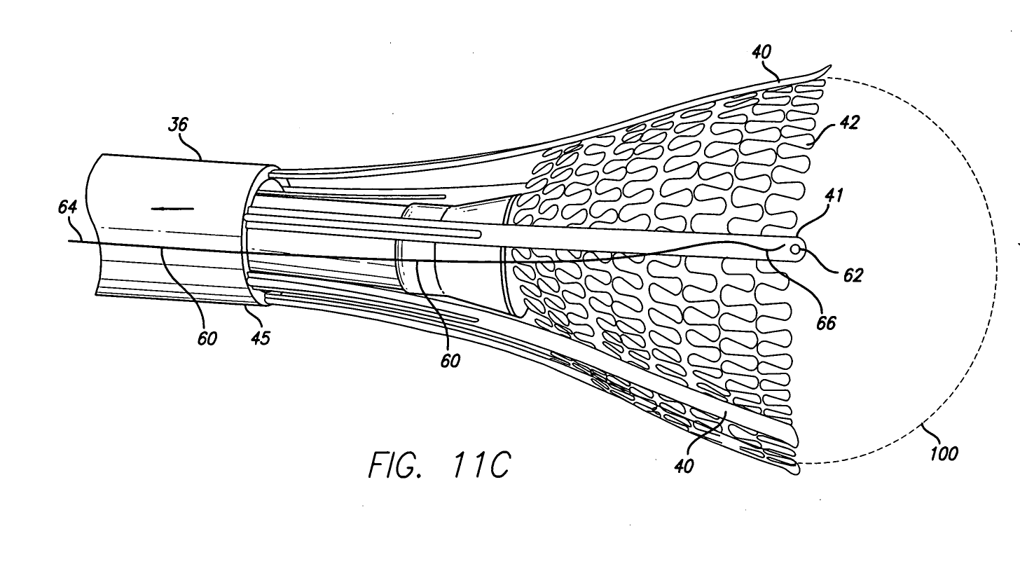

In at least one embodiment, as shown in FIGS. 11A-11C the doubled lines are advantageously used to advance the harness 42 onto the heart and then to easily disconnect the line from the harness when so desired by the surgeon. When one free end 64 or 66 of the line is pulled, the other free end of the line will slide through the harness 42, and the harness 42 will be released from the line 60. When both free ends 64 and 66 of the line 60 are pulled, the harness 42 will stay connected with the line 60 and a pulling force in a distal direction towards the heart will be exerted on the harness 42 by the line 60. Each line 60 thereby forms a detachable connection with the cardiac harness 42.

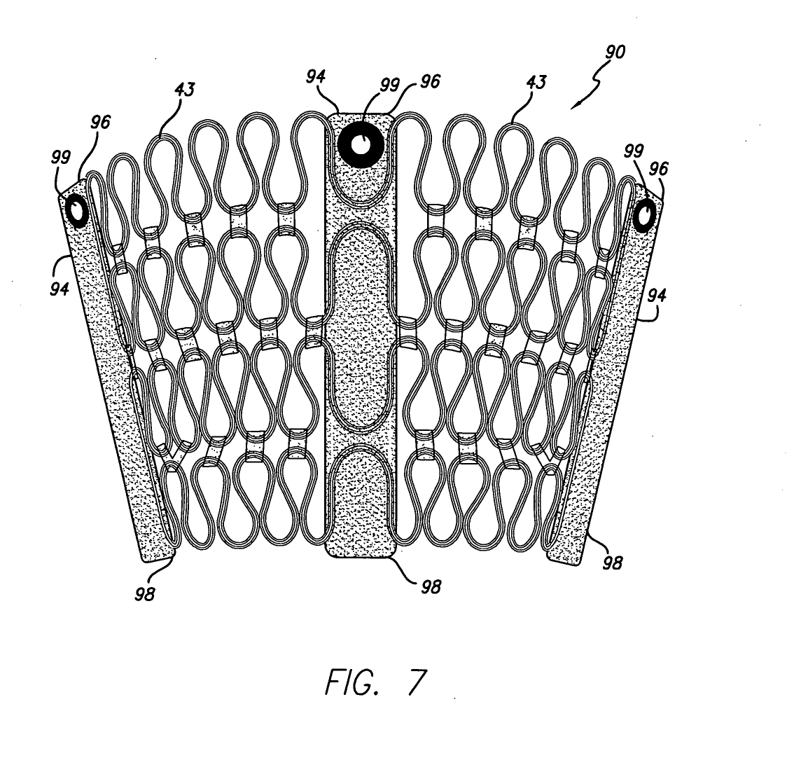

In at least one embodiment, as shown in FIG. 7, a cardiac harness 90 includes struts 94 which advantageously reinforce the harness 90. The struts 94 are useful when pulling the harness 90 onto the heart. The struts may be elongated substantially flat members formed, for example, from a biocompatible implantable polymer material such as a silicone rubber or plastic. In another embodiment, the struts may be reinforced with a metal, for example, stainless steel or titanium. In still another embodiment, the struts may be reinforced with a biocompatible plastic or silicon. The struts are highly flexible, but more rigid than the rows of undulating elastic members 43 of the harness. In at least one embodiment, the struts of the cardiac harness are molded into the cardiac harness. The struts 94 have a distal end 96 and a proximal end 98. Each strut 94 has at least one reinforced eyelet 99 on the distal end 96 for the threading of the line 60 (FIG. 8) therethrough. The eyelets 99 may be treated with or made from biocompatible materials having a low coefficient of friction. In at least one embodiment, the eyelets 99 are positioned nearer to the distal ends of the struts 94. The struts 94 permit the pulling forces to be transferred onto the harness 90 by the lines 60 while minimizing the risk of tearing of the undulating elastic members 43 by the line 60. Furthermore, because pulling directly on the undulating elastic members 43 is avoided, the struts also are advantageous in preventing distortion of the shape of the harness by the pulling action. The struts also help distribute the pulling forces of the lines uniformly across the rows of undulating elastic members of the harness.

Referring now also to FIG. 8, the cardiac harness 42 is loaded into the housing cavity 44 for delivery onto the heart. The cardiac harness is connected with the lines 60, before or after the cardiac harness is disposed in the housing cavity. The cardiac harness is oriented within the housing cavity so that it can be properly disposed onto the heart with the base of the harness facing distally. In other words, the cardiac harness is positioned in the housing cavity with the largest opening perimeter of the harness oriented distally towards the superior aspect of the heart. Each doubled line 60 is slidingly connected with the friction reducing member 62 on a pull rod 40. The free ends 64 and 66 of each doubled line 60 are then brought back proximally towards the handle 32. The lines 60 are preferably kept organized to avoid the lines 60 becoming tangled.

Referring briefly again to FIG. 4, in at least one embodiment, a plurality of line guides 70 may be included in the wall of the housing 36. Although the line guides 70 as illustrated in this embodiment are located on the inside of the housing wall, in other embodiments the line guides 70 may be located on the outside of the housing wall or within the housing wall. The line guides 70 are axially oriented channels or grooves in the wall of the housing 36 that slidingly retain the lines 60 (FIG. 8) and help keep the lines from tangling. One or more lines 60 may pass through each line guide 70. In at least one embodiment, there are at least two line guides 70 for each pull rod 40. This is advantageous in avoiding tangling of the lines 60 by keeping each strand of the doubled line 60 separated in its own line guide 70. In one embodiment the line 60 is permitted to axially slide through the line guides 70 during deployment of the pull rods 40, whereby the pull rods 40 may be deployed around the heart without displacing the harness 42 from the housing cavity 44.

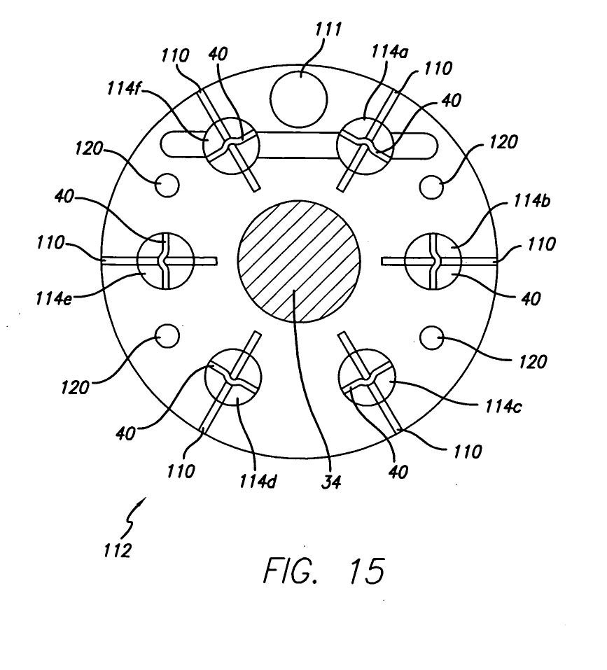

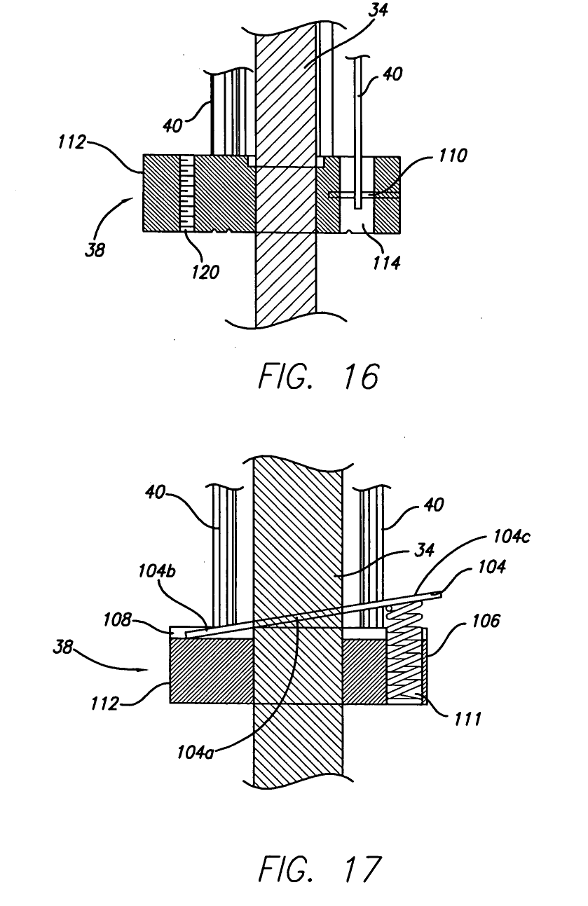

With reference next to FIGS. 14 and 17, at least one embodiment of the delivery device 30 includes a control assembly 38. As indicated above, the control assembly 38 is movable axially relative to the shaft 34 of the delivery device 30. Preferably, the control assembly 38 includes a position-retaining arrangement, such as a friction brake assembly 102, for example. The friction brake assembly 102 is configured to permit the control assembly 38 to be selectively retained in a desired position relative to the shaft 34. Preferably, the friction brake assembly 102 is configured to be easily actuatable, along with movement of the control assembly 38, by one hand of a user of the device 30.

The illustrated friction brake assembly 102 includes a brake element 104 and a biasing member, such as a spring 106. The brake element 104 includes an annular central portion 104a surrounding the shaft 34. Opposing end portions 104b, 104c extend in an outward direction from the central portion 104a substantially opposite from one another. The first end portion 104b is retained within a channel 108 of the control assembly 38, for example, by a pin (not shown).

The spring 106 is retained within a cavity 111 and is arranged to bias the second end 104c of the brake element 104 away from the control assembly 38. Preferably, the spring 106 biases the brake element 104 such that an inner diameter-defining surface of the central portion 104a is in frictional contact with the shaft 34 so as to secure the control assembly 38 in a desired position relative to the shaft 34. The brake element 104 may be pivoted toward the control assembly 38 by pushing the end 104c toward the control assembly 38 to disengage the brake element 104 from the shaft 34 and permit relative movement between the control assembly 38 and the shaft 34. In another embodiment, two such brake elements 104 are provided. However, each brake element is oriented to pivot in an opposite direction. As such, one brake element better prevents distal movement of the assembly relative to the shaft, and the other brake element better prevents proximal movement of the assembly relative to the shaft.

With reference to FIGS. 14-17, in one embodiment the control assembly 38 includes a substantially cylindrical body portion 112. A plurality of passages, generally referred to by the reference numeral 114, extend axially through the body portion 112 of the control assembly 38. In the illustrated embodiment, the passages 114 are substantially cylindrical in shape and are generally equally distributed in a circular arrangement coaxial with the shaft 34. Preferably, the passages 114 are generally aligned with corresponding channels 50 (FIG. 4) formed in the housing 36.

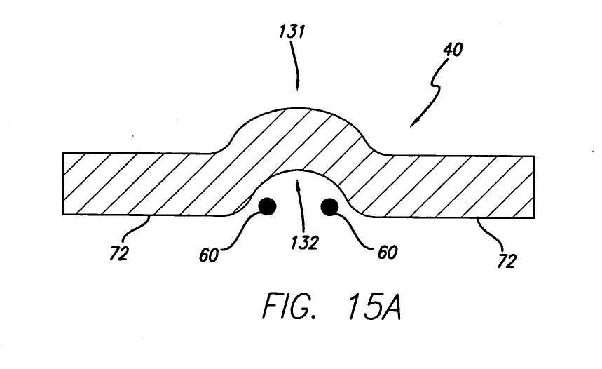

Referring more specifically now to FIGS. 14-15, a cover 116 is fixed to a proximal end of the body portion 112. The cover 116 closes a proximal end of the passages 114 and the cavity 111. A plurality of fasteners, such as screws 118, engage corresponding threaded apertures 120 of the body portion 112 to secure the cover 116 to the body portion 112. Furthermore, in at least one embodiment, a plurality of retaining slots or clips 33 (FIG. 14) may be included with the handle 32 for retaining ends 64, 65, and 66 of the lines 60. In at least one embodiment the retaining slots or clips 33 may be conveniently positioned around the perimeter of the cover 116. The retaining slots or clips 33 may be advantageous in organizing the free ends of the lines 60.

With reference now to FIG. 15, in one embodiment, the body portion 112 includes six passages 114, referred to specifically by the reference numerals 114a-114f. As a matter of convenience, the passages 114a-114f are referred to herein by their relative positions as depicted in FIG. 15. As such, passages 114a and 114f comprise an upper pair of passages, passages 114b and 114e comprise a central pair of passages, and passages 114c and 114d comprise a lower pair of passages. Passages 114b-114f are distributed in a clockwise direction around passage 114a, and are equally spaced in relationship to one another.

With reference particularly to FIGS. 15 and 16, each of the above-described passages 114a-f are configured to receive a proximal end of one of the pull rods 40. The pull rods 40 are secured within their respective passages 114a-f by a shaft or pin 110 passing through an opening (not shown) within the pull rod 40 and may be supported by the body portion 112 of the control assembly 38. Thus, as described above, the pull rods 40 are fixed for axial movement with the control assembly 38.

In the illustrated embodiment, the pull rods are supported generally in the center of the passages 114a-f, with their respective inner surfaces arranged generally tangentially to the center axis of the shaft 34. In addition, with reference also to FIG. 15A, in at least one embodiment, a center portion 131 of each pull rod 40 is generally semicircular in cross-section such that the inward facing surface 72 defines a recess 132. The recess 132 in each pull rod may be configured to accommodate at least one line 60 (FIG. 8). Therefore, in at least one embodiment, the lines can pass at least a part of the way from distal to proximal along the delivery device 30 within the recesses 132. In some embodiments this is an alternative to, or in addition to, the lines 60 passing through retaining slots or clips 33 disposed on the handle 32. The recesses may also be an alternative to, or in addition to the lines 60 passing axially through the line guides 70 in the housing 36. In one embodiment, the lines 60 may continue proximally from the recesses 132 through passages (not shown) in the cover 116 to exit the control assembly 38.

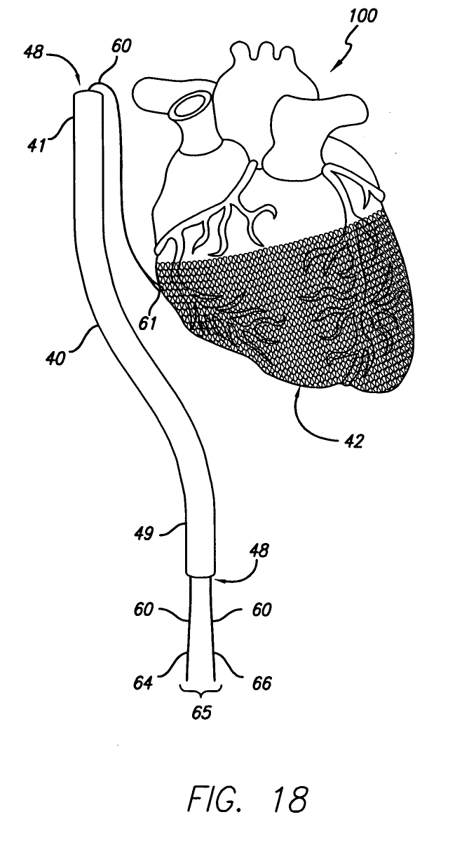

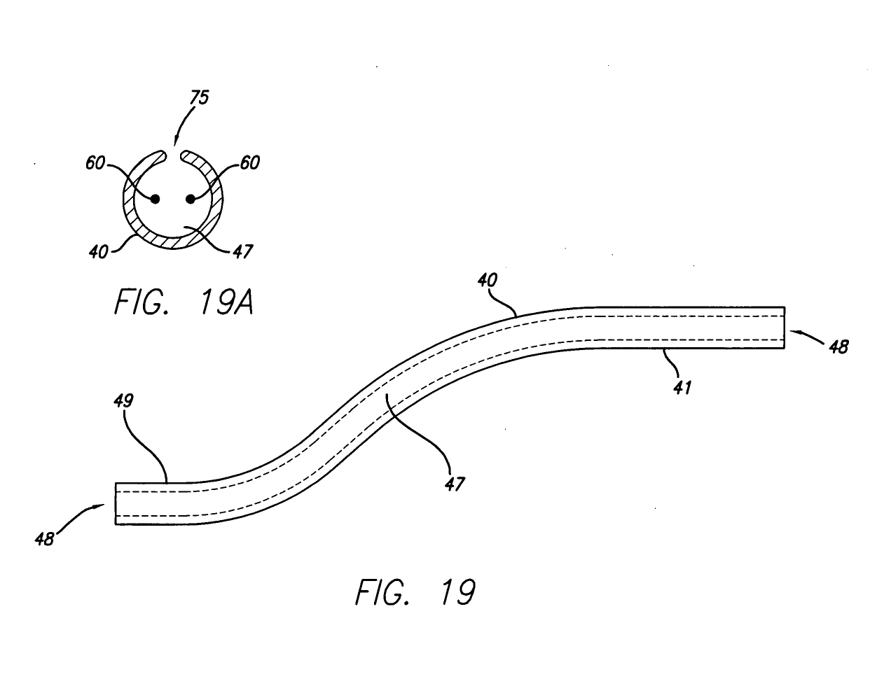

Referring now to FIGS. 18, 19, and 19A, at least one additional embodiment of the invention includes a contoured pull rod. The distal end 41 of the pull rod 40 is shaped or contoured to fit into the pericardial space. Furthermore, in at least one embodiment, the pull rod 40 has a hollow inner channel 47 with opposing openings 48 on either end. The line 60 may positioned into the channel 47 through the opening 48 on the distal end 41, traverse through the hollow inner channel 47, and exit the pull rod 40 through the opening 48 located at the proximal end 49 of the pull rod. In at least one embodiment, the pull rod 40 may further include a side opening 75 (FIG. 19A) in the wall extending along the length of the hollow inner channel 47. The side opening 75 is advantageous in inserting the line 60 into the inner channel 47 through the side opening 75. The side opening thereby avoids the need to insert the pairs of free ends 65 of the line 60 into the distal 41 opening 48 and thread the free ends to the proximal 49 opening 48. The pull rod 40 may further include a friction reducing member 62 on at least one end 41 and 49. As described above, a doubled line 60 may be slidingly connected with the harness 42. Pulling on the paired free ends 65 disposes the harness 42 on the heart. Pulling on either free end 64 or 66 alone detaches the doubled line 60 from the harness 42.

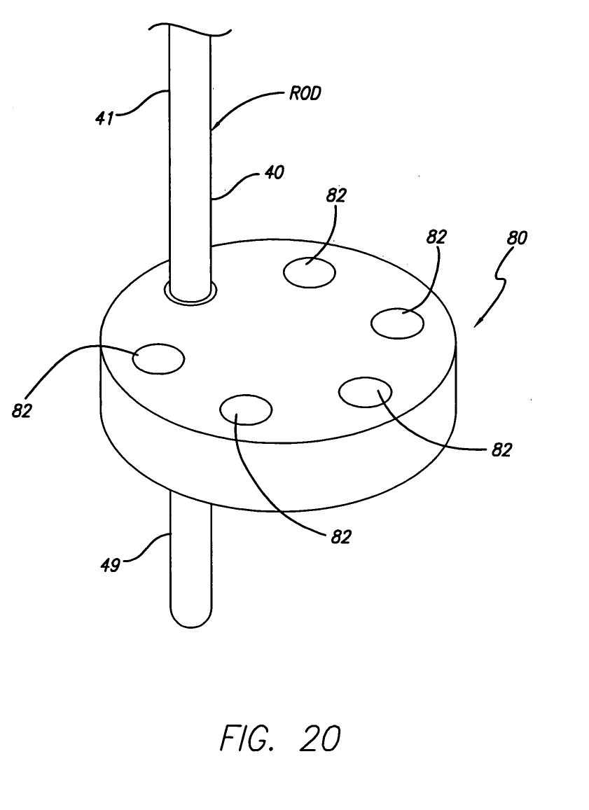

In at least one embodiment, the pull rods 40 may be individually disposed into the pericardial space. Referring now also to FIG. 20, before or after the distal portions 41 of the pull rods 40 are disposed in the pericardial space, the proximal portions 49 of the pull rods may be disposed onto and retained by a carrier 80. In at least one embodiment, the carrier has a plurality of channels 82 for receiving the pull rods 40. In at least one embodiment, the proximal portions 49 of the pull rods 40 and the channels 82 may have a similarly shaped cross sectional appearance, for example, an oval, square, triangle, polygon, trapezoid, or the like, wherein the pull rods 40 may be more securely retained within the channels 82. Furthermore, in at least one embodiment the proximal end 49 of the pull rod 40 has a tapered end configured to be retained within a tapered channel 82 in the carrier 80. A shaped or tapered proximal end 49 of the pull rod 40 and cylindrical channel 82 may limit the amount of axial movement of the pull rod 40 through the channel 82. A shaped proximal end 49 of the pull rod 40 and cylindrical channel 82 may also limit the amount of rotation of the pull rod 40 within the channel 82. For example, an oval or hexagonal shape to the proximal end 49 of the pull rod 40 and cylindrical channel 82 would prevent rotation of the pull rod 40 in the cylindrical channel 82. Other alternative methods of retaining a pull rod 40 in a carrier 80, such as using clips, pins, springs, and the like could be used, and are well known to those in the art.

Patients suffering from congestive heart failure often are at risk to additional cardiac failures, including cardiac arrhythmias. When such arrhythmias occur, the heart must be shocked to return it to a normal cycle, typically by using a defibrillator. Implantable cardioverter/defibrillators (ICD's) are well known in the art and typically have a lead from the ICD connected to an electrode implanted in the right ventricle. Such electrodes are capable of delivering a defibrillating electrical shock from the ICD to the heart. However, other cardiac devices have placed the electrodes on the epicardium at various locations, including on or near the epicardial surface of the right and left heart. These devices also are capable of distributing an electrical current from an implantable cardioverter/defibrillator for purposes of treating ventricular defibrillation or hemodynamically stable or unstable ventricular arrhythmias. The invention is therefore useful for delivering a cardiac harness having electrodes for treatment of heart disease by pacing, cardioversion, or defibrillation.

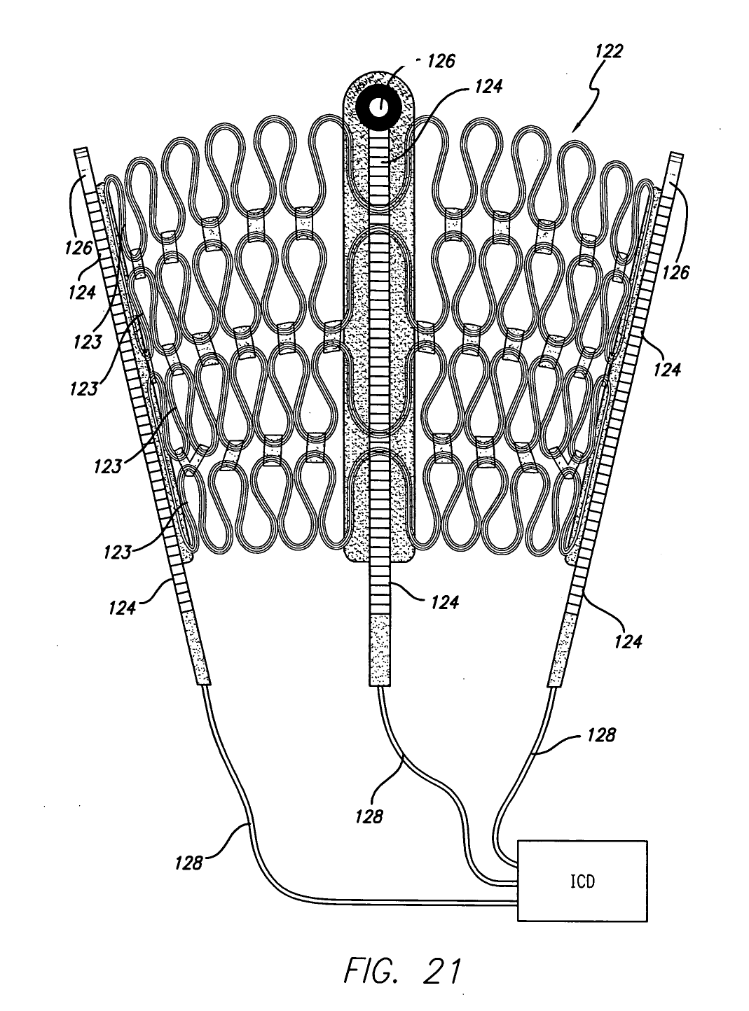

Referring briefly now to FIG. 21, in yet at least one other embodiment, the invention includes a cardiac harness 122 having electrodes 124 connected with a power source for use in defibrillation or pacing and a method of applying the harness 122 having electrodes 124 onto the heart. An example of such a harness is described in U.S. patent application Ser. No. 10/704,376 the entire contents of which are incorporated herein by reference. The cardiac harness 122 includes a number of rows or strands of undulations 123. The harness 122 further includes coils or electrodes 124. The rows or strands of undulations 123 may be interconnected together so that the harness 122 can flex and can expand and retract circumferentially. The undulations 123 of the cardiac harness 122 may be coated with a dielectric coating to electrically insulate the undulations 123 from an electrical shock delivered through at least one electrode 124. Further, the electrodes 124 may be at least partially coated with a dielectric material to insulate the electrodes 124 from the cardiac harness 122. In one embodiment, the strands or rows of undulations 123 are formed from Nitinol and are coated with a dielectric material such as silicone rubber. Lines 60 (FIG. 8) may be detachably connected to the harness 122 as previously described with other embodiments of the harness 42 and 90. In at least one embodiment, the harness 122 includes reinforced eyelets 126 for connection of the lines 60 to the harness. The eyelets 126 may also be made from a dielectric material such as silicone rubber and attached with or near to the electrodes 124. The electrodes 124 may be electrically connected with wires 128. The wires 128 may be connected to an ICD for example a pacemaker or defibrillator including a power source (not shown).

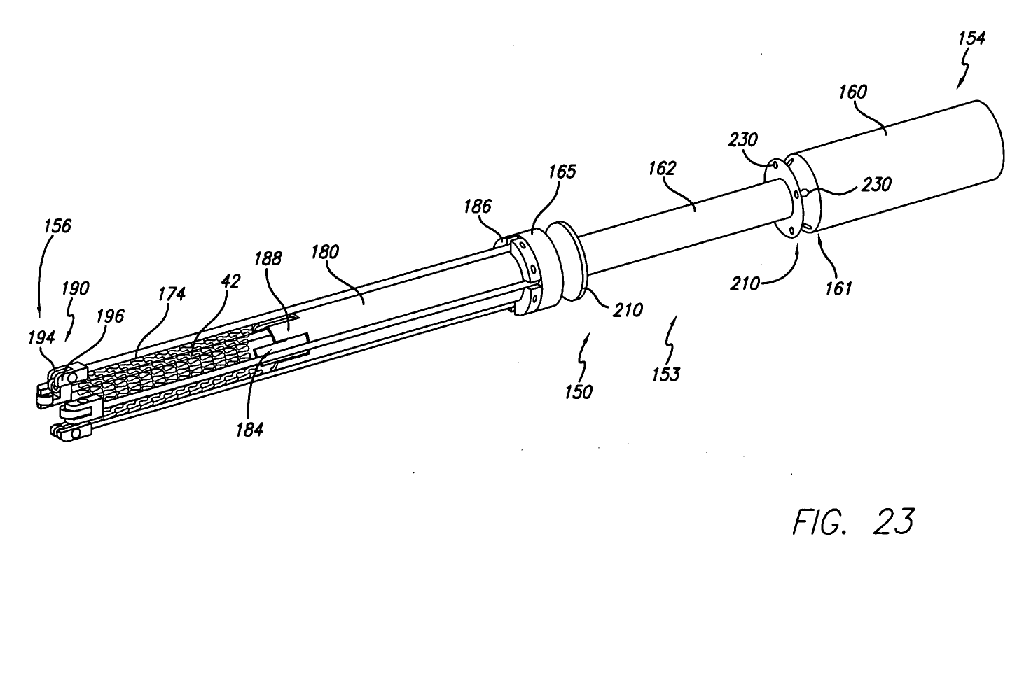

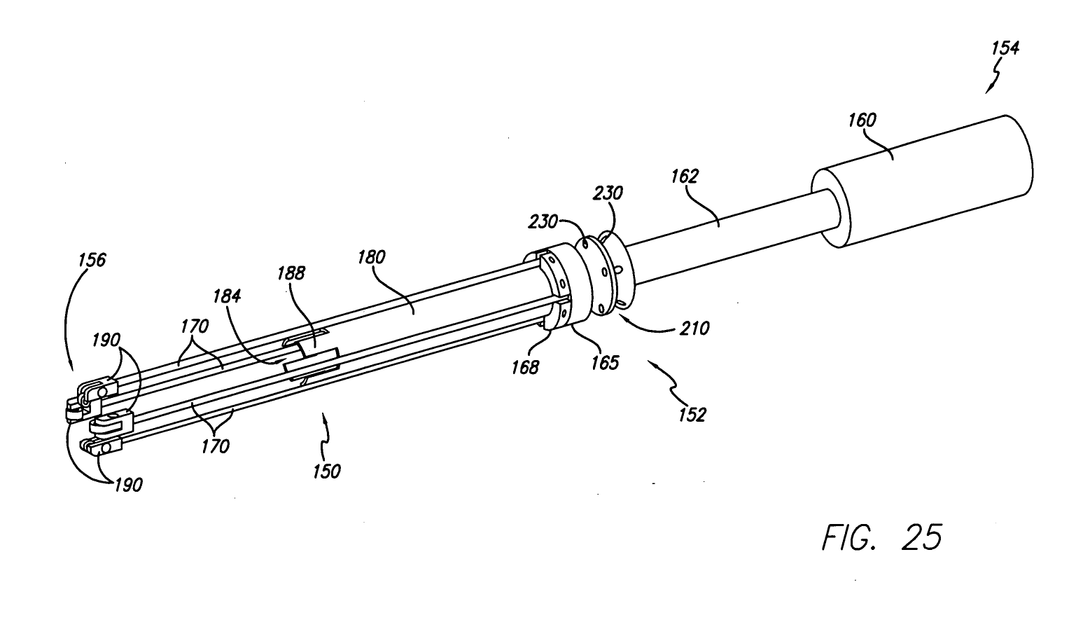

Referring now to FIGS. 22-23 and FIG. 25, yet another embodiment of the cardiac harness delivery device 150 is shown. The delivery device has a first undeployed configuration 151 (FIG. 22), a second intermediate configuration 152 (FIG. 25), and a third fully deployed configuration 153 (FIG. 23). The delivery device has a proximal end 154 and a distal end 156. The proximal end of the delivery device includes a handle 160 connected to the proximal end of a shaft 162.

The delivery device 150 further includes a housing 180. The housing has a housing cavity 184 for retaining a cardiac harness 42, 122 (FIG. 21) therein, the cardiac harness including any CRD configured for mounting on the heart. The proximal end of the housing includes a housing base 186 that is connected to the distal end of the shaft 162. In one embodiment, the shaft extends beyond the housing base and the shaft may further include a positioning member 52 and suction tube 54 (FIG. 1).

The cardiac harness delivery device may further include a pull rod slider 165 that has a central channel 166 that is configured to slidingly articulated with the shaft 162 that passes generally centrally therethrough. In one embodiment, the pull rod slider may further include the control assembly 38 or the friction brake assembly 102 (FIGS. 14, 16-17). Disposed around the periphery of the pull rod slider are a plurality of peripheral openings 168, each configured to retain a proximal end 172 of a pull rod 170.

The pull rods 170 are slidingly articulated with the housing 180, 36 (FIG. 4). In at least one embodiment, the pull rods are slidingly articulated within housing channels 50 (FIG. 4) that are disposed around the periphery of the housing. In one embodiment the housing channels are disposed on the outside of the housing. In another embodiment, the housing channels are disposed on the inside of the housing. In yet another embodiment, the housing channels may be disposed within the wall of the housing. The housing channels may be dove shaped channels 50 (FIG. 4) to retain correspondingly shaped rods, as described in detail elsewhere herein for another embodiment. In one embodiment, there are four pull rods. In other embodiments, there may be fewer or greater number of pull rods. In at least one embodiment, the number of pull rods are in the range of four to twelve pull rods.

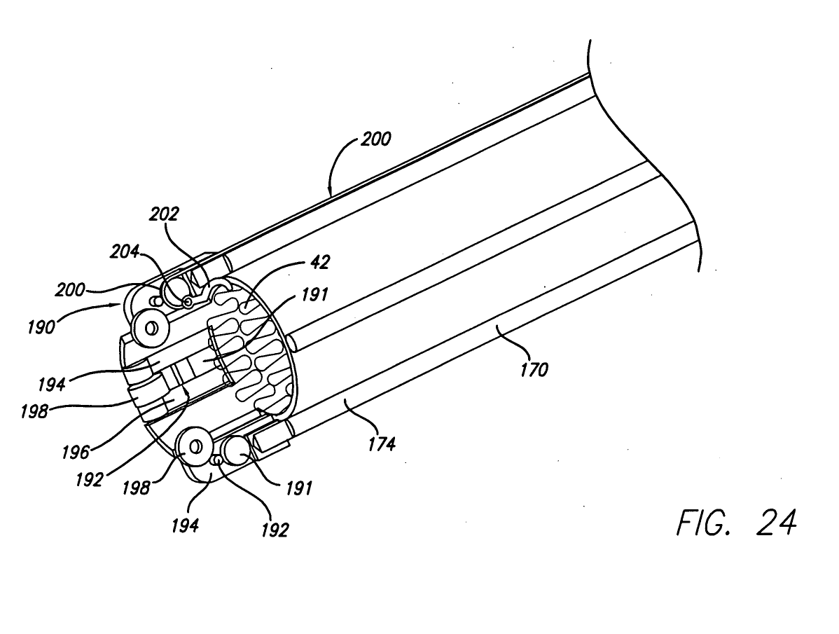

Referring now also to FIG. 24, the distal ends 174 of the pull rods 170 include rod heads 190 that are configured to permit a line 60 or a cable 200 to slide therethrough. The rod heads may be injection molded into the distal ends of the pull rods, or manufactured independent of the pull rod and connected to the pull rod with snap fittings, adhesives, rivets, or other connection methods known in the art. The rod heads may include a friction reducing member 62 configured for sliding of the line or cable therewith. In one embodiment, the rod heads include a first wall 194 and a second wall 196. In one embodiment, each rod head further includes at least a first guide pin 191 that is cylindrical in configuration, and further having a longitudinal axis that is generally perpendicular to the walls of the rod head. In a preferred embodiment, each rod head further includes a second guide pin 192 that is also cylindrical in configuration and has a longitudinal axis that is perpendicular to the walls of the rod head. In one embodiment, the second guide pin is distal to the first guide pin. In one embodiment, the second guide pin is smaller in diameter than the first guide pin. A cable may be positioned over the top or distal periphery of the first guide pin which functions as the friction reducing member. In one embodiment, the cable may be positioned over the top or distal periphery of the first guide pin and further positioned between the first guide pin and the second guide pin, thereby assisting in slidingly retaining the cable at the distal end 174 of the pull rod 170. In one embodiment, the rod head further includes an epicardial roller 198 that reduces friction between the rod head and the heart, as the pull rod 170 is advanced over the heart. In other embodiment, a flat surface with a low coefficient of friction may be substituted for the epicardial roller or used with the epicardial roller. In yet other embodiments, alternative sliding or rolling members known in the art that provide a decreased coefficient of friction between the rod and the heart may be included on the distal end of the rod.

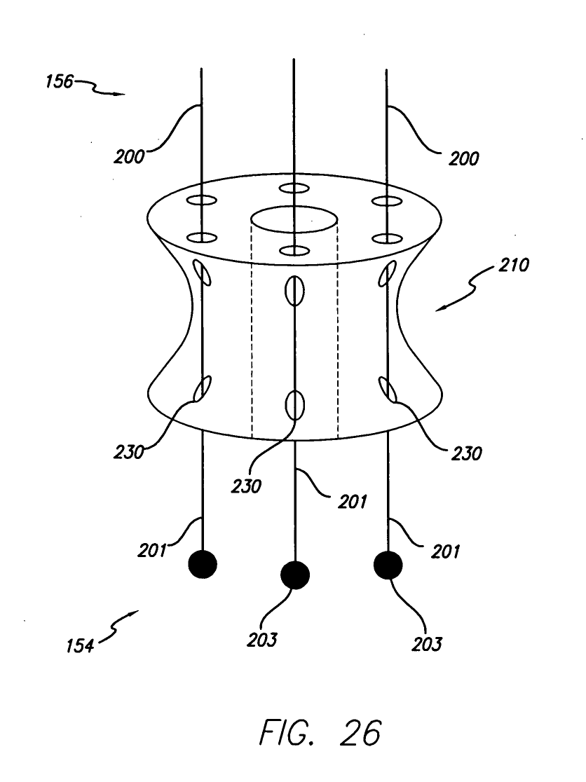

Referring still to FIG. 24, in one embodiment, clasps 202 are disposed on the distal end of the cables 200, the clasps being configured to detachably connect with the harness 42. The clasps, for example, may be blunt hooks that insert into the harness through undulations 43, 123 (FIGS. 6A-6D, 7, 21) or insert into the eyelets 99, 126 provided on the harness 122 (FIGS. 7, 21). The proximal ends 201 of the cables are disposed on the cable retention members 230 of the cable slider 210 (FIG. 26). In one embodiment, the proximal ends of the cables may be slidingly disposed on the cable retention members. In another embodiment the cables may be securely gripped by the cable retention members, as described in more detail herein. In still other embodiments, the loop 61 of doubled lines 60 (FIGS. 11A-11C), as described elsewhere herein, may be connected to the harness and easily removed after mounting the harness on the heart.

Referring also now to FIG. 26, in one embodiment, the delivery device 150 further includes a cable slider 210 that is slidingly disposed on the shaft 162 through a central opening 220 in the cable slider. Disposed around the periphery of the cable slider is a plurality of cable retention members 230, for example, holes, configured to hold the cables 200 or lines 60. The cable retention members may also be, for example, clips, hooks, hook and loop fasteners, cleats, or tubes. In at least one embodiment, the cable retention members are configured to allow the cables to slide through the cable retention member. In at least another embodiment, the cable retention members are configured to grip the cables, wherein the cables cannot slide through the cable retention members. Other possible configurations known in the art for slidingly holding a cable or securely gripping a cable may also be used as cable retention members.

Each cable 200 may be configured with a sufficient amount of length or slack to allow the pull rods 170 to fully extend into the intermediate second configuration 152 without pulling the harness 42 out of the housing cavity 184. In one embodiment, each cable 200 has a length approximately equal to the distance from the center of the first guide pin 191 to the distal extent 161 of the handle 160, the distance being measured when the delivery device 150 is in the fully deployed third configuration 153 (FIG. 23). The cable length is configured whereby the harness will not be deployed out of the housing 180 in the intermediate second configuration 152 (FIG. 25), where both the pull rod slider 165 and the cable slider are both disposed distally on the shaft 162, generally adjacent to the housing base 186. The cable length is further configured whereby the harness will be fully deployed out of the housing 180 in the fully deployed third configuration 153 (FIG. 23), where the pull rod slider is disposed distally on the shaft generally adjacent to the housing base and the cable slider is disposed proximally on the shaft generally adjacent to the handle 160. In the undeployed first configuration 151 (FIG. 22), the cable 200 will be slack. The slack part of the cable may be disposed in the housing cavity 184 or disposed outside of the housing 180 or alongside of the shaft 162. In the intermediate second configuration 152 (FIG. 25), the cable will be taut and ready to pull the harness towards the distal end of the rods. In the fully deployed third configuration 153 (FIG. 23), the cable will remain taut and the harness will have been pulled out of the housing and will be positioned at the distal end of the rods.

Referring again to FIG. 26, in one embodiment, the cable 200 is permitted to slide proximally 154 through the cable retention members 230. In one embodiment, the proximal end of the cable includes an end cap 203 that keeps the proximal end 201 of the cable from sliding distally 156 out of the cable retention member. In another embodiment, the cable is gripped securely by the cable retention members, which may include grips, clips, snug openings, or other configurations known in the art for securing a line or cable.

Referring specifically now to FIG. 22, in the undeployed first configuration 151, the pull rod slider 165 and the cable slider 210 are disposed proximally on the shaft 160, wherein they are generally adjacent to each other, and the handle 160. In the first configuration, the cable slider is disposed on the shaft generally adjacent to the handle. Furthermore, the pull rod slider is disposed on the shaft generally adjacent to the cable slider. Also, the harness 42 is disposed in the housing cavity 184. In the first configuration, the distal ends 174 of the pull rods 170 are disposed adjacent to the distal opening 188 in the housing 180.

Referring specifically now to FIG. 25, in the intermediate extended configuration 152, the pull rod slider 165 and the cable slider 210 are both disposed distally on the shaft 160, and generally adjacent to each other. In the intermediate configuration, the pull rod slider is disposed on the shaft generally adjacent to the housing base 186. The harness 42 remains disposed in the housing cavity 184. The distal ends 174 of the pull rods 170 are now in a fully extended configuration, wherein the distal ends of the pull rods are maximally extended beyond the distal opening 188 of the housing 180 and are configured to be deployed upon the heart. The cables 200 (not shown) are connected with the harness (not shown). In one embodiment the clasps 202 (FIG. 24) on the distal end of the cables are connected with the harness. The other ends of the cables are disposed on the cable retention members 230 of the cable slider. In the intermediate extended configuration, the slack in the cable has been taken up, preparing the harness for deployment out of the housing.

Referring specifically now to FIG. 23, in the fully deployed third configuration 153, the pull rod slider 165 is disposed distally on the shaft 160, and the cable slider 210 is disposed proximally on the shaft 160. The pull rod slider is disposed on the shaft generally adjacent to the housing base 186. The cable slider is disposed on the shaft generally adjacent to the handle 160. There is substantially no slack in the cables 200 (not shown). The harness 42 has been deployed out of the housing cavity 184 towards the distal ends 174 of the pull rods 170, wherein the harness may be mounted upon the heart. The distal ends of the pull rods remain in the fully extended configuration, wherein the distal ends of the pull rods are extended beyond the distal opening 188 of the housing 180 and are configured to be deployed around the heart. In one embodiment, the clasps 202 (FIG. 24) on the distal end of the cables are connected with the harness. In yet another embodiment, the loops 61 of the doubled line 60 (FIGS. 11A-11C), are connected with the harness (FIGS. 6A-6D). In one embodiment, the proximal ends 201 of the cables are securely gripped by the cable retention members 230 of the cable slider. In another embodiment, the proximal ends of the cables have slid distally in the cable retention members, wherein the end caps 203 (FIG. 26) are adjacent to the cable retention members. In at least one embodiment, the cable may be disconnected from the harness after the harness is mounted on the heart. In yet another embodiment, the harness may remain connected with the cables or lines, and the cables or lines cut to release the harness from the connection to the delivery device 150.

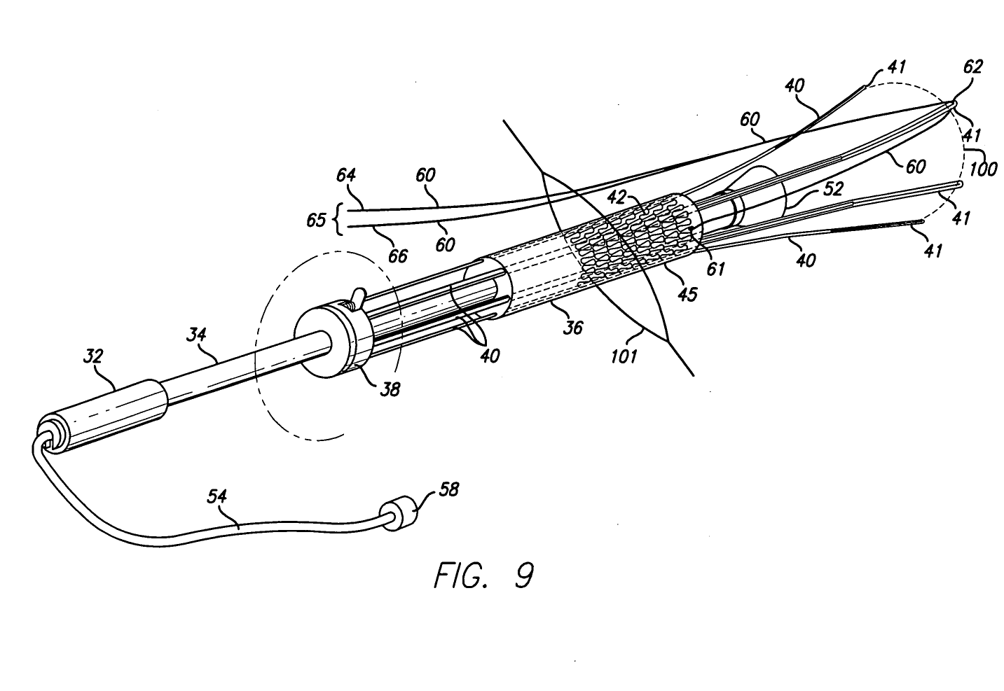

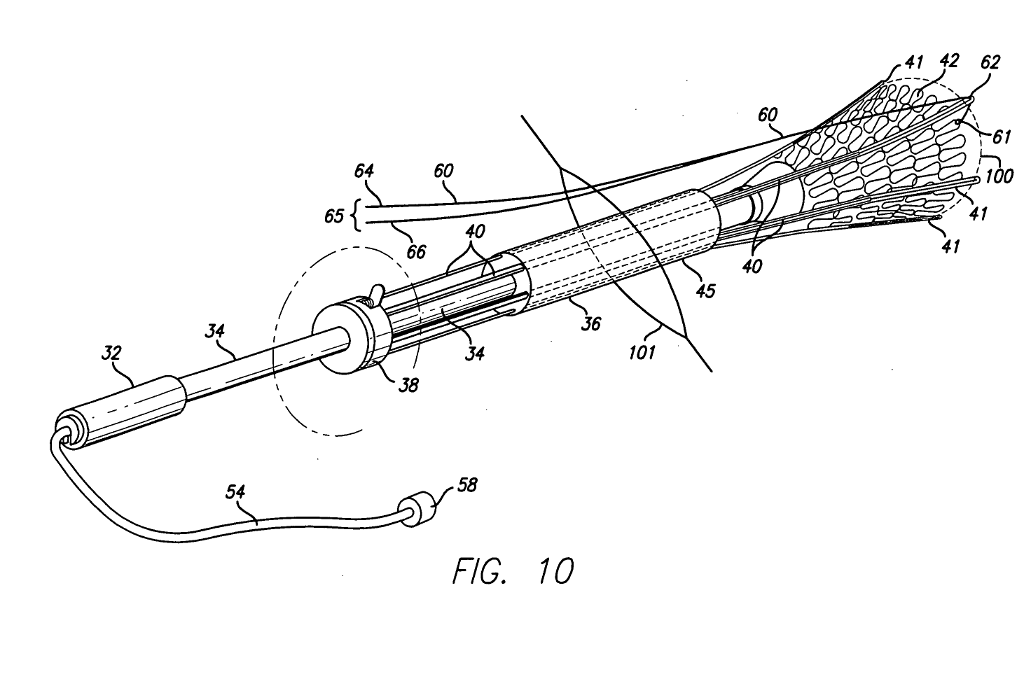

Referring now to FIGS. 9-12, the invention further includes a method of disposing a cardiac harness 42 or 90 onto the heart. The method comprises making a subcostal, intercostal, or subxiphoid incision to access the mediastinum, making an incision in the pericardium (not shown), introducing the distal end 31 (FIG. 1) of the delivery device 30 into the patient through the incisions, and advancing a plurality of pull rods 40 over the surface of the heart 100 between the epicardium and the pericardium. In one embodiment the method includes pulling on lines 60 connected with the harness to advance the harness towards the distal ends 41 of the pull rods 40 until the harness is in a desired location on the heart. The ends 65 of the lines 60 are pulled proximally, in a caudal or infracardiac direction, to mount the cardiac harness 42 or 90 on the heart. Pulling in a caudal or infracardiac direction means pulling in a direction from the heart towards the umbilicus. In one embodiment the method includes pulling on the paired ends 65 of doubled line 60. The method may further include pulling on one free end 64 or 66 of each pair 65 of free ends of doubled line 60 until all lines are disconnected from the harness. The pull rods 40 are then withdrawn from around the heart 100 while leaving the harness in place on the heart. The method may further include removing the delivery device 30 from the patient, and closing the incision. The method may also include delivering a harness having electrodes 124 (FIG. 21).

In at least one embodiment the method further includes connecting a plurality of lines 60 with the harness 42 or 90, by threading one free end 64 or 66 of the line through the harness or an intermediary connecting member 68 (FIG. 6D). In one embodiment, the lines 60 are preferably connected around the distal periphery of the harness, or in other words, the base of the harness 42. The method further includes doubling each line over itself to form a loop 61 located near the connection of the harness to the line 60, loading the cardiac harness into the housing cavity 44 of the delivery device 30, and slidingly connecting each doubled line with the distal portion 41 of a pull rod 40. The method may further include bringing pairs 65 of the free ends of the lines 60 back towards the proximal end of the delivery device 30. In at least one embodiment, the method further includes slidingly connecting the lines 60 with the line guides 70 in the housing 36 (FIG. 4).

In at least one embodiment the method includes connecting the cardiac harness 42 or 90 with at least one line 60, connecting the line 60 with the distal portion 41 of at least one pull rod 40, and disposing the at least one pull rod 40 upon the heart 100. The method also includes pulling the cardiac harness 42 or 90 into position on the heart by pulling proximally on the line 60 in an infracardiac direction, disconnecting the cardiac harness from the at least one pull rod, and withdrawing the at least one pull rod from the pericardial space. In at least one embodiment, the method is used for disposing a harness 122 including electrodes 124 (FIG. 21).

In at least one embodiment the method includes connecting a cardiac harness 42 or 90 with the distal portions 41 of a plurality of pull rods 40, disposing each pull rod 40 upon the heart 100, disposing the pull rods onto a pull rod carrier 80, pulling the cardiac harness 42 or 90 into position on the heart, disconnecting the cardiac harness 42 or 90 from the pull rods, and withdrawing the pull rods 40 from the pericardial space.

In at least one embodiment the method further includes closing the pericardium after disposing the harness 42 or 90 onto the heart 100. In at least one embodiment, the method further includes slidingly connecting each doubled line 60 with the distal end 41 of a pull rod 40 by slidingly disposing the doubled line onto a friction reducing member 62 (FIGS. 5A-D). In at least one embodiment, the method includes the cardiac harness and lines being preassembled with the delivery device 30 by the manufacturer or prior to delivery to the user. Furthermore, although the methods that have been described relate to minimally invasive surgery, it will readily be recognized by those skilled in the art that the present invention may also be used during open chest heart surgery in a similar manner.

Referring now to FIG. 9, in at least one embodiment, a minimally invasive surgical incision 101 is made, including an opening into the pericardium (not shown). The distal end 45 of the housing 36 of the delivery device 30 is introduced through the surgical incision 101 and into the pericardial sac. The pull rods 40 are slowly advanced between the epicardium and pericardium until the pull rods 40 surround the heart 100 (schematically illustrated). The lines 60 may be allowed to slide on friction reducing members 62 until the pull rods 40 are fully deployed in position around the heart 100. The cardiac harness 42 may preferably be maintained inside the housing cavity 44 until the pull rods 40 are fully deployed in position around the heart 100.

Referring now also to FIG. 10, the cardiac harness 42 is disposed onto the heart 100 (schematically illustrated) by pulling in concert together on the paired 65 free ends 64 and 66 of each of the double over lines 60. All of the pairs 65 of free ends 64 and 66 of doubled over lines 60 may be pulled at once, or each pair 65 may be pulled individually. Although only one doubled line is shown in FIG. 10, it should be understood that in one preferred embodiment, a plurality of doubled lines are disposed on a plurality of pull rods 40. The pairs 65 may be pulled randomly or in sequence to advance the harness onto the heart. Pulling in concert on the pair 65 of free ends 64 and 66 of the doubled over line 60 causes the harness 42 to be pulled out of the housing cavity 44 and towards the distal end 41 of the pull rod 40. The harness 42 is slowly advanced over the heart 100 (schematically illustrated) until the harness is mounted onto the heart.

Referring now to FIGS. 11A-11C, the cardiac harness 42 is mounted onto the heart 100 (schematically illustrated) by pulling on pairs 65 of free ends 64 and 66 of each doubled line 60. The cardiac harness 42 is then disconnected from each of the lines 60 by pulling on only one free end 64 or 66 of each doubled line 60 until the line 60 is fully out of the harness 42. As shown in FIG. 11B, as one free end 64 is pulled proximally, the other free end 66 is allowed to move distally. As shown in FIG. 11C, as the one free end 64 is pulled even further proximally, eventually the other free end will slide free of any connection with the harness 42 or the friction reducing member 62. The configuration of disposing the doubled line 60 on the harness 42 described above is advantageous, because when pulling on either one free end 64 or 66 or the pair 65 of free ends 64 and 66, the forces are always directed towards deploying the harness 42 towards the distal end of the pull rod 40. This configuration may therefore prevent the harness 42 from being pulled off of the heart 100 when disconnecting the lines 60 from the harness 42.

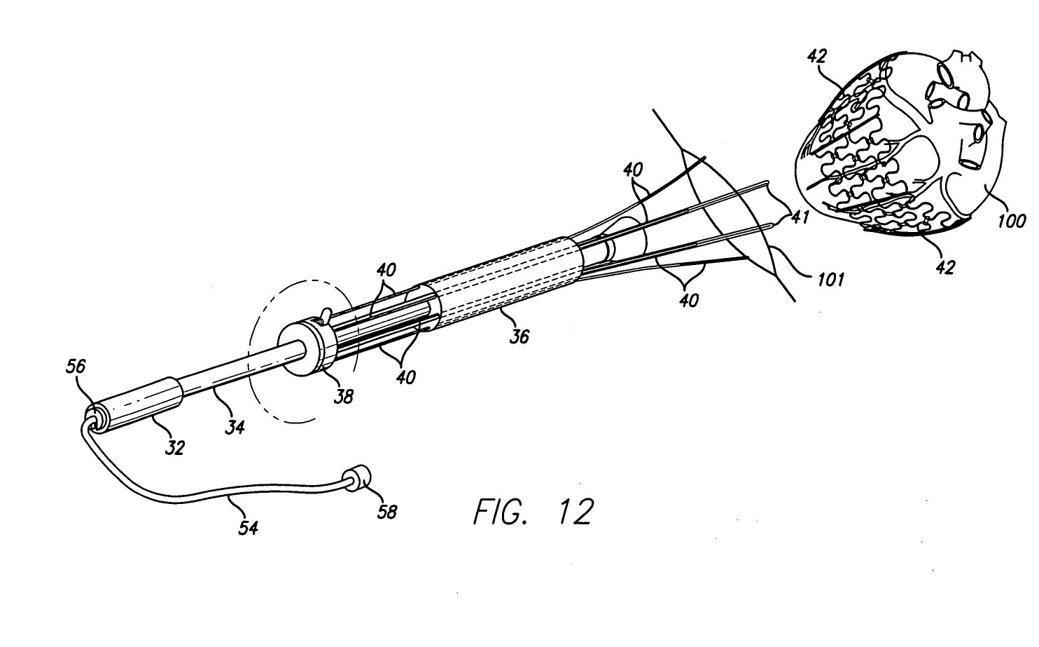

Referring now to FIG. 12, once the cardiac harness 42 has been positioned on a patient's heart, the control assembly 38 is retracted relative to the shaft 34 such that the plurality of pull rods 40 are also retracted relative to the cardiac harness 42 and the heart 100. Upon retraction of the pull rods 40, relative motion may be experienced between the inner surface 72 (FIG. 13) of the pull rods 40 and the cardiac harness 42. That is, the inner surface of the pull rod 40 slides along the cardiac harness 42 along a withdrawal path away from the heart 100. The pull rods 40 are withdrawn off of the harness 42 and off of the heart 100. Referring now to FIG. 13 and also FIG. 3, in at least one embodiment, the distal portion 41 of the pull rod 40 is configured with a divergent angle from the central axis of the delivery device 30. This divergent angle is advantageous in minimizing forces exerted by the inner surface 72 of the pull rod 40 against the harness, such forces tending to drag the harness off of the heart during retraction of the pull rods. Therefore, once the cardiac harness 42 is properly positioned on the heart, retraction of the pull rods 40 tends to not disturb the positioning of the harness 42 on the heart.

The present invention also includes a method of delivering the cardiac harness 122 with electrodes 124 onto the heart, by pulling the harness onto the heart. In at least one embodiment, a method of disposing the cardiac harness with electrodes onto the heart comprises detachably connecting a cardiac harness 122 having electrodes 124 onto lines 60 (FIG. 8). As shown in FIGS. 9-12, pull rods 40 are disposed into the pericardial space and around the epicardial surface of the heart 100. The method further includes pulling on the lines 60 to dispose the harness 122 between the epicardium and pericardium onto the heart, detaching the harness 122 from the lines 60, and removing the pull rods 40 from the epicardial space. The method may further include electrically connecting the electrodes with an ICD, for example a pacemaker or a defibrillator.

Referring again to FIGS. 22-25, the invention further includes a method of mounting a harness upon the heart including providing a cardiac harness delivery device 150 having an undeployed first configuration 151, an intermediate second configuration 152, and a fully deployed third configuration. The cardiac harness 90, 122 is disposed in the housing cavity 184 of the delivery device. The cardiac harness is connected with clasps 202 on the end of cables 200. The method further includes inserting the distal end 156 of the delivery device into an incision made in a patient, the delivery device being in the first configuration. The method further includes sliding the pull rod slider 165 and the cable slider 210 distally, wherein the distal ends 174 of the pull rods 170 are disposed around the heart. The method also includes sliding the cable slider proximally to the distal end 161 of the handle 160, wherein the cardiac harness is deployed out of the housing cavity and mounted upon the heart. In one embodiment, the method further includes disconnecting the harness from the cable clasps and removing the delivery device from the patient.

The invention may be embodied in other forms without departure from the spirit and essential characteristics thereof. The embodiments described therefore are to be considered in all respects as illustrative and not restrictive. Although the present invention has been described in terms of certain preferred embodiments, other embodiments that are apparent to those of ordinary skill in the art are also within the scope of the invention. Accordingly, the scope of the invention is intended to be defined only by reference to the appended claims.

What is claimed:

1. A cardiac harness delivery device for use with pulling a cardiac harness onto a heart, comprising:
a handle slidingly connected with a proximal end of an elongate shaft;
a housing affixed to a distal end of the elongate shaft, the housing having a housing cavity configured to hold a cardiac harness therein; and
a plurality of pull rods, having a proximal end disposed on the handle and a distal end configured for slidingly retaining at least one line.

2. The cardiac harness delivery device of claim 1, wherein the pull rods have an axially sliding connection with the housing.

3. The delivery device of claim 1, further including a plurality of shaped channels disposed in the housing wall, wherein the shaped channels are configured for sliding axial movement with the pull rods.

4. The cardiac harness delivery device of claim 1, further including friction reducing members disposed on the distal end of the pull rods.

5. The cardiac harness delivery device of claim 1, further including lines slidingly disposed on the distal end of the pull rods.

6. The cardiac harness delivery device of claim 5, further including a cardiac harness releasably connected with the lines.

7. The cardiac harness delivery device of claim 1, further including a plurality of line guides disposed in the housing.

8. A method of mounting a cardiac harness onto the heart, comprising:
releasably connecting a cardiac harness with a plurality of lines;
disposing the lines on a cardiac harness delivery device having a plurality of pull rods;
making at least one incision configured for access to a heart of a patient;
introducing a distal end of the cardiac harness delivery device into the patient through the incision;
advancing the plurality of pull rods over the heart;
mounting the cardiac harness on the heart by pulling on the lines;
disconnecting the lines from the harness; and
removing the pull rods from around the heart.

9. The method of claim 8, wherein the lines are disposed on a distal end of the pull rods.

10. The method of claim 8, wherein the lines are disposed on friction reducing members disposed on a distal end of the pull rods.

11. The method of claim 10, wherein the harness is connected to a loop in the line, and the harness is disconnected from the line by pulling on one of a pair of free ends of the line.

12. The method of claim 8, further including:
loading the cardiac harness into a housing cavity of the cardiac harness delivery device; and
disposing free ends of the lines at a proximal end of the cardiac harness delivery device.

13. The method claim 8, further including slidingly connecting the lines with line guides in a housing.

14. The method of claim 8, further including closing a pericardial incision after mounting the cardiac harness on the heart.

15. The method of claim 8, further including loading the cardiac harness within a housing and disposing the lines onto the pull rods prior to delivery to the surgeon.

16. A method of disposing a cardiac harness onto a heart comprising:
disposing a cardiac harness on a line;
disposing the line on a distal end of a pull rod;
disposing the pull rod into a pericardial space of a patient;
mounting the cardiac harness on the heart by pulling on the line in a caudal infracardiac direction;
disconnecting the cardiac harness from the pull rod; and
withdrawing the pull rod from the pericardial space.

17. The method of claim 16 wherein the line is a doubled line, and the cardiac harness is slidingly disposed on a loop in the doubled line.

18. The method of claim 17, wherein the cardiac harness is mounted on the heart by pulling on a pair of free ends of the doubled line.

19. The method of claim 17, wherein the cardiac harness is disconnected from the pull rod by pulling on one free end of a pair of free ends of the doubled line.

20. The method of claim 16, further including disposing the pull rods onto a pull rod carrier.

21. The method of claim 16, further including disposing the line through an inner channel in the pull rod.

22. The method of claim 21, wherein the line is disposed through the inner channel in the pull rod by inserting the line through a side opening in the inner channel.

23. The method claim 16, further including connecting electrodes disposed on the harness to an implantable cardioverter defibrillator.

24. A cardiac harness delivery device, comprising:
an elongate shaft having a proximal end and a distal end;
a housing disposed on the distal end of the elongate shaft, the housing having a housing cavity configured to deployingly hold a cardiac harness therein;
a pull rod slider slidingly disposed on the shaft, the pull rod slider being movable between a position proximal on the shaft and another position distal on the shaft;
a cable slider slidingly disposed on the shaft proximal to the pull rod slider, the pull cable slider being movable between a position proximal on the shaft and another position distal on the shaft;
a plurality of pull rods slidingly connected with the housing and having a proximal end disposed on the pull rod slider and a distal end configured for slidingly connecting with a plurality of cables.

25. The cardiac harness delivery device of claim 24, further including a handle disposed on the proximal end of the elongate shaft.

26. The cardiac harness delivery device of claim 24, further including a plurality of cable retention members disposed around a periphery of the cable slider.

27. The cardiac harness of claim 24, further including the plurality of cables, wherein the cables are slidingly disposed distally on the distal ends of the pull rods and the cables are disposed proximally on the cable slider.

28. The cardiac harness delivery device of claim 27, wherein the plurality of cables are slidingly disposed in the cable retention members, the proximal ends of the cables further including end caps.

29. The cardiac harness delivery device of claim 27, wherein the proximal ends of the cables are gripped by the cable retention members.

30. The cardiac harness delivery device of claim 27, further including clasps disposed on the distal ends of the cables, the clasps configured to detachably connect with the harness.

31. The cardiac harness delivery device of claim 24, further including rod heads disposed on the distal ends of the rods.

32. The cardiac harness delivery device of claim 24, further including friction reducing members disposed on the distal ends of the rods.

33. A method of mounting a cardiac harness on a heart of a patient, comprising:
providing a cardiac harness delivery device having an undeployed first configuration, an intermediate second configuration, and a fully deployed third configuration;
disposing a cardiac harness in a housing cavity of the delivery device;
detachably connecting the harness with distal ends of cables;
inserting a distal end of the delivery device into the patient, the delivery device being in the first configuration;
sliding a pull rod slider and a cable slider distally, wherein the delivery device assumes the intermediate second configuration and distal ends of pull rods are disposed on the heart; and
sliding the cable slider proximally to a distal end of a handle, wherein the delivery device assumes the third configuration and the cardiac harness is pulled out of the housing cavity and pulled onto the heart.

34. The method of claim 33, further including disconnecting the harness from the cables and removing the delivery device from the patient.
